(19) United States
(12) Patent Application Publication
(10) Pub. No.: US 2026/0294964 A1
(43) Pub. Date: Oct. 1, 2026

(54) PHARMACEUTICALS CONTAINING CHELATORS WITH STRONG ZN IONS-HOLDING FEATURE FOR THE TREATMENT OF SYMPTOMS AND DISEASES CAUSED BY MICROORGANISMS/DISEASES CAUSED BY TOXINS OR NEUROTOXINS/CANCER/SEVERAL SYNDROMES

(71) Applicant: Cong Nhan HUYNH, Ho Chi Minh City (VN)

(72) Inventor: Cong Nhan HUYNH, Ho Chi Minh City (VN)

(21) Appl. No.: 19/479,950

(22) PCT Filed: Apr. 26, 2024
(86) PCT No.: PCT/IB2024/054083

§ 371 Date: Oct. 29, 2025

(30) Foreign Application Priority Data
May 5, 2023 (VN) 1-2023-02967

(51) Int. Cl. A61K 33/30; A61K 47/54

(52) U.S. Cl. CPC A61K 33/30; A61K 47/542

(57) ABSTRACT

Metalloproteins on the surface of pathogenic microorganisms have Zn ion-high density, these Zn ions help pathogenic microorganisms to bind to metal ions on metalloproteins on host cell surface (similar to Zn ions find and bind to other metal ions in electric batteries with Zn-other metal electrode pairs). Pathogenic microorganisms and their fragments release Zn-rich products into the host body, these Zn-rich products rapidly make chelators with Zn ions-holding feature (which are mainly derivative of polyamines/ aminopolycarboxylic acids) fully hold Zn ions, deplete these active chelators, when active chelators are depleted, Zn-rich products bind to host metalloproteins, generating most symptoms, then host body provide new active chelators which separate Zn-rich products from host metalloproteins, repelling most symptoms. Drugs of invention contains chelators with strong Zn ion-holding feature (which are derivative of polyamines/aminopolycarboxylic acids) that have the following effects:—rapidly reduces most symptoms;—weaken microorganisms and loses their ability to bind to host cells;—strengthen host immune system;—clears the accumulation of Zn-rich products from host metalloproteins, repelling most symptoms;—repelling post-disease syndrome;—and so on. Drugs of invention contains [high active chelators+agent that creates strong physical effects (in treated tissues)] to treat diseased tissues (such as bacterial infection tissues/necrotic tissues/fungal infection tissues/benign tumors/cancerous tumors) with outstanding effectiveness.

### FIELD OF INVENTION

The invention belongs to the pharmaceutical field containing a chelating agent with the ability to retain [Zn ions/other heavy metal ions] to treat symptoms of diseases caused by pathogenic microorganisms and to prevent or treat diseases caused by pathogenic microorganisms/diseases caused by toxins and neurotoxins/diseases that weaken the immune system due to chronic diseases or due to the decline of polyamine derivatives in the elderly/cancer/some other diseases/some syndromes/diseased tissues (such as infection tissues/necrotic tissues/tissues containing many toxins/benign tumors/cancerous tumors).

### DESCRIPTION

### Prior Arts

Until now, due to biology/medicine does not know that [Zn/other transition metal]-rich products released from pathogenic microorganisms and their fragments (referred to as [Zn/other transition metal]-rich products from pathogenic microorganisms) rapidly deplete chelators with [Zn ion/other transition metal ions] holding feature (still activity to hold [Zn ion/other transition metal ion]), means depleting chelators that play the role in resisting symptoms in the host body (host is animal/human), and under conditions where the host is depleted of chelators with [Zn ions/other transition metal ions]-holding feature, products rich in [Zn/other transition metals] from pathogenic microorganisms attaches to the host's metalloproteins, causing various symptoms (such as fatigue/runny nose/sputum thickening/diarrhea/dyspnea . . . )/neurological symptoms (such as sore throat/cough/soft tissue pain/muscle pain/joint pain/bone tissue pain/eye pain/headache/blood thick/tissue fluid thick/alveolar fluid thick/blood clots/cytokine storm/respiratory failure/acute respiratory failure . . . ), and produces the long-term post-illness syndromes; and products rich in [Zn/other transition metals] from pioneering pathogenic microorganisms are sources of Zn/other transition metals that cause secondary pathogenic microorganisms to multiply rapidly and cause cancer cell and cancerous tissue to rapidly multiply and to rapidly increase in size, and weaken the patient's immune system during the depletion of chelators with [Zn ions/other transition metal ions]-holding feature, and the depletion of chelators with [Zn ions/other transition metal ions]-holding feature in the body of patients with certain chronic diseases (such as HIV/tuberculosis/other chronic diseases caused by pathogenic microorganisms/stage 3 and 4 cancer) weaken the immune system of these patients in long-term period. And until now, biology/medicine has not known the decline of chelators with [Zn ions/other transition metal ions]-holding feature in the elderly, causing the elderly's immune system to weaken over time; and polyamine derivatives in the body of obese people in the form of fatty soap polyamine salts, which are inactive forms of holding Zn ions/other metal ions, weakening the immune system of obese people, so when obese people suffering from diseases caused by pathogenic microorganisms, the symptoms are intense and the pathogenic microorganisms multiply rapidly, causing the diseased condition of obese patients to quickly worsen/lead to death easily.

And until now, due to biology/medicine does not know that chelators with [Zn ions/other transition metal ions]-holding feature are agents that create a series of effects as outlined below to help the host body to recover quickly, so up to now, there are no pharmaceuticals containing chelators with [Zn ions/other transition metal ions]-holding feature are to prevent or to treat symptoms/diseases as mentioned above; and so far biology/medicine does not know when to introduce [agents that produce strong physical effects+chelators with [Zn ions/other transition metal ions]-holding feature]+without agent that creates the foam structure for diseased tissue/with agent that creates the foam structure for diseased tissue] (or with the addition of other therapeutic active ingredients, collectively referred to as chelator and other therapeutic active ingredients as therapeutic active ingredients) into diseased tissue (such as infection tissue/necrotic tissue/fungal infection tissue/tissues containing high viscous fluid/benign tumors/cancerous tumors), it increases the dispersion speed of the active ingredients in the treated tissue by tens to hundreds of times, thereby making the tight structure of the diseased tissues (such as structures of benign tumors/cancerous tumors/fungal-infection tissues)/structures containing high viscous fluid (such as infection tissue/necrotic tissue) that have lost their protective capacity for [pathogenic entities (such as pathogenic microorganisms/cancer cells)/pathogenic agents (such as toxins and nutrients for pathogenic microorganisms/cancer cells)] within the diseased tissues, means that the strong physical effects/[strong physical effects+foam structure] in the treated tissue help to with the tight structure/high viscous fluid-filled structure of the diseased tissues which are the structures that make the treating diseased tissues difficult; so up to now, there are no pharmaceuticals with the ingredients [agent that produces strong physical effects+chelator with [Zn ions/other transition metal ions]-holding feature/[agent that produces strong physical effect+chelator with [Zn ions/other transition metal ions]-holding feature+agents that create the foam structure for diseased tissue] to treat the above diseased tissues.

And until now, due to biology/medicine does not know that chelators with [Zn ions/other transition metal ions]-holding feature are agents that help separate toxins/neurotoxins (the structure of toxins/neurotoxins needs to hold [Zn ions/other transition metal ions] to attach to host metalloproteins to cause poison) from the host's metalloproteins, neutralizing the toxicity of toxins/neurotoxins produced by pathogenic microorganisms/from outside the host body, so up to now, there are not pharmaceuticals whose active ingredients are chelators with [Zn ions/other transition metal ions]-holding feature to treat diseases caused by toxins/neurotoxins produced by pathogenic microorganisms/from the outside entering the host body. And biology/medicine does not know that the decline of polyamine derivatives in the elderly body make to increase the accumulation of Zn ions/other transition metal ions in tissues/nervous tissues/brain tissues, generate the dementia syndrome/tremor syndrome/blurred eyes syndrome, so up to now there are not pharmaceuticals whose active ingredients are chelators with strong [Zn ions/other transition metal ions]-holding feature to treat for the elderly as well as help the elderly's immune system to strengthen. And biology/medicine does not know that chelators with strong [Zn ions/other transition metal ions]-holding feature reduce the voltage of metalloproteins of living entities in the patient's body, which occurs when the patient uses these drugs, makes when patients use other pharmaceuticals at the same time as chelators with strong [Zn ions/other transition metal ions]-holding feature, it greatly increases the effectiveness of other drugs, thereby greatly reducing the dose of the other pharmaceuticals need to use . . .

Until now, due to biology/medicine has not known the vital roles of chelators with strong [Zn ions/other transition metal ions]-holding feature in the biological world (including in the host body (host is animal/human)), because until now biology/medicine has not known the vital roles of chelators with strong [Zn ions/other transition metal ions]-holding feature in the biological world (including in the host body (host is animal/human)), and biology/medicine with unknown major changes involved [Zn/other transition metal] and involved chelators with [Zn ions/other transition metal ions]-holding feature when the host suffers from various diseases, and biology/medicine do not know the valuable benefits of physical effects when created in the patient's body/in diseased tissue, contributing to greatly increase the effectiveness of treatment of the various diseases/making greatly to increase the effectiveness of treatment the diseased tissues, so until now there are no pharmaceuticals containing chelators with strong [Zn ions/other transition metal ions]-holding feature to treat many different diseases, so to date there are no pharmaceuticals containing [chelators with strong [Zn ions/other transition metal ions]-holding feature+agent producing strong physical effects]/[chelators with [Zn ions/other transition metal ions]-holding feature+agent that produces strong physical effects in the treated tissue+agent that creates the foam structure for the treated tissue] for the treatment of various diseases/for the treatment different diseased tissues. And biology/medicine does not know that synthetic chelators are polyamine aminopolycarboxylate/Mg aminopolycarboxylate/Ca aminopolycarboxylate/salts made from polyamine with acid with the sulfur functional group in the molecule are substances that have the ability to quickly relieve symptoms/help the patient's body recover quickly,

and the ability of the above synthetic chelators to quickly eliminate symptoms/help the patient's body recover quickly are greater than the abilities of natural chelators that present in the host body (mainly are histidine/lysine/arginine derivatives) dozens to hundreds of times, so until now there are no pharmaceuticals containing the above synthetic chelating agents to treat different symptoms/treat different diseases.

More specifically, up to now, due to biology/medicine does not know many things related to [Zn ion/other transition metal ions] and related to chelators with [Zn ions/other transition metal ions]-holding feature. and related to physical effects, leading to the situation that there are no pharmaceuticals whose active ingredients are [chelators with strong [Zn ions/other transition metal ions]-holding feature]/there are no pharmaceuticals whose active ingredients are chelators with strong [Zn ions/other transition metal ions]-holding feature+agent that produces strong physical effects]/there are no pharmaceuticals whose active ingredients are [chelators with strong [Zn ions/other transition metal ions]-holding feature+agent that produces strong physical effects+agents that produce strong physical effects+agents that produce tiny gas bubbles] for the treatment of various diseases/various syndromes/weakened immune systems/diseases caused by toxins and neurotoxins/cancers/diseased tissues . . . , specifically, until now biology/medicine does not know the following, and up to now, the following situations exist:

Agents and conditions that produce symptoms in diseases caused by microorganisms, and the situation that pharmaceuticals to treat the symptoms in the way to eliminate the agents that cause the symptoms:

Until now, due to biology/medicine does not know the agents and conditions that produce symptoms in diseases caused by microorganisms, such as various symptoms/different neurological symptoms/blood thickening or tissue fluid thickening or alveolar fluid thickening/blood clots/cytokine storm/respiratory failure/acute respiratory failure due to [Zn/other transition metal ion]-rich products from pathogenic microorganisms and their fragments (referred to as Zn/other transition metal ion]-rich products from pathogenic microorganisms) are released into the host body, these [Zn/other transition metal ion]-rich products adhere to metalloproteins of host cells/tissues/nerve tissue/brain tissue, causing the above-mentioned symptoms, especially in the condition that the patient's body has low quantity of chelators with [Zn ions/other transition metal ions]-holding feature (these chelators are mainly polyamine derivatives such as histidine/lysine/arginine/putrescine/cadaverine/spermidine . . . ) or depleted of these chelators (because these chelators already fully hold Zn ions/other transition metal ions), so up to now, there are no pharmaceuticals that helps separate the Zn-rich products/other transition metal-rich products as mentioned above from the host's metalloproteins from the metalloproteins of the host's cells/tissues/nerve tissues/brain tissues, and to eliminate these Zn-rich products/other transition metal-rich products from the host body through urine (there are only pharmaceuticals that treat symptoms by reducing the perception of symptoms (such as reducing the perception of tissue pain/muscle pain/headache).

Agents and conditions that cause pathogenic microorganisms to multiply rapidly (including pioneer pathogenic microorganism/secondary pathogenic microorganism), and the situation of pharmaceuticals that inhibit/kill these pathogenic microorganisms:

Until now, due to biology/medicine does not know the factors that cause secondary microorganisms to multiply rapidly in the patient's body, due to Zn/other transition metal-rich products from pathogenic microorganisms causing secondary microorganisms to obtain Zn/other transition metal raw materials, and deplete chelators with [Zn ions/other transition metal ions]-holding feature, and under conditions where the host body is depleted of chelators with [Zn ions/other transition metal ions]-holding feature,

secondary pathogenic microorganisms can easily utilize these Zn/other transition metal-rich products from pathogenic microorganisms (pioneer pathogenic microorganism) as a source of Zn/other transition metals for secondary pathogenic microorganisms to multiply rapidly, so up to now, there are no pharmaceuticals with the active ingredients being chelators with [Zn ions/other transition metal ions]-holding feature, to inactivate Zn/other transition metal-rich products from pathogenic microorganisms (causing pioneer disease) for secondary pathogenic microorganisms cannot multiply.

Agents and conditions that cause severe symptoms in obese people/elderly people/people with underlying diseases when suffering from diseases caused by microorganisms, and the situation of pharmaceuticals to treat diseases caused by microorganisms in obese people/elderly people/people with underlying diseases:

Until now, due to biology/medicine does not know the body of obese people, polyamine derivatives in the form of aliphatic soap salts that have very weak ability to hold Zn ions/other transition metal ions, therefore, when suffering from diseases caused by pathogenic microorganisms, the body of obese people lacks highly active polyamine derivatives to prevent symptoms from occurring; and the bodies of elderly people/people with underlying diseases have low levels of polyamine derivatives/[low polyamine derivatives that still have the ability of holding Zn ions/other transition metal ions], causing elderly people/people with underlying diseases when suffer from diseases caused by microorganisms having severe symptoms, so until now there are no pharmaceuticals with the active ingredients being chelators with [Zn ions/other transition metal ions]-holding feature to help the elderly/people with underlying diseases avoid severe symptoms when suffering from diseases caused by pathogenic microorganisms.

Agents and conditions that weaken immune systems in several diseases, and pharmaceutical situation that treat weakened immune systems:

Until now, due to biology/medicine does not know that the microorganisms that cause chronic diseases such as HIV/tuberculosis/other chronic diseases/cancer tissues in stage 3.4 are biological factories that release Zn/other transition metal-rich products which enter the host body continuously, leaving the host body in the condition of low quantity chelators with [Zn ions/other transition metal ions]-holding feature aways, causing the lack of these chelator to integrate into the Cu ions/Mg ions/Ca ions of the structure of antibacterial alkaline peptides/antibody proteins/microbicidal proteins inside leukocytes, makes antibacterial alkaline peptides/antibody proteins/microbicidal proteins inside leukocytes to reduce the ability to kill pathogenic microorganisms, and lacks chelators with [Zn ions/other transition metal ions]-holding feature to envelope Zn ions/other transition metal ions on the surface of pathogenic microorganisms/metastatic cancer cells that have left the cancer tissue to prevent pathogenic microorganisms/metastatic cancer cells that have left the cancer tissue to attach to host metalloproteins to cause disease/to cause metastasis to healthy tissues, so up to now, there is no medicine with the active ingredient being chelators with [Zn ions/other transition metal ions]-holding feature to strengthen the weakened immune systems as mentioned above.

Conditions that weaken the immune systems of the elderly, and pharmaceutical situation that strengthen the immune systems of the elderly; and situation that cause dementia syndrome/tremor syndrome/cataract syndrome in the elderly, and pharmaceuticals to treat these syndromes:

Until now, due to biology/medicine does not know that the elderly body gradually reduces the amount of polyamine derivatives which are chelators with [Zn ions/other transition metal ions]-holding feature, so over time Zn/other transition metal-rich products from food/from pathogenic microorganisms increasingly accumulate in metalloproteins in the tissues/eye tissue/nervous tissues/brain tissue of the elderly, weakening the immune system of the elderly, and causing the appearance of dementia syndrome/tremor syndrome/cataract syndrome with the condition getting worse over time, so up to now, there are no pharmaceuticals with the active ingredient being chelators with [Zn ions/other transition metal ions]-holding feature to strengthen the immune system of the elderly and there are no pharmaceuticals to effectively treat dementia syndrome/tremor syndrome/cataract syndrome in the elderly.

Conditions and agents that cause dementia syndrome/tremor syndrome/cataract syndrome in the elderly, and pharmaceutical situation to treat these syndromes:

Until now, due to biology/medicine does not know the amount of polyamine derivatives, which are chelators with [Zn ions/other transition metal ions]-holding feature in the body of the elderly decrease over time, make the increasing of the accumulation of [Zn ions/transition metal ions] in cells/tissues/nerve tissues/brain tissue, and make the increasing of the accumulation of [Zn ions/transition metal ions] in the cells/tissues/eye tissues/nerve tissues/brain tissues, giving rise to dementia syndrome/tremor syndrome/cataract syndrome in the elderly, so up to now, there are no pharmaceuticals with the active ingredient being chelators with [Zn ions/other transition metal ions]-holding feature to treat dementia syndrome/tremor syndrome/cataract syndrome in the elderly in the way that eliminates the above-mentioned accumulations.

Conditions and agents that weaken the immune system in the elderly, and the situation of pharmaceuticals to treat the weakened immune system in the elderly:

Until now, due to biology/medicine does not know the amount of polyamine derivatives which are chelators with [Zn ions/other transition metal ions]-holding feature in the body of the elderly gradually decrease over time, makes the increasing of the accumulation of [Zn ions/transition metal ions] in cells/tissues/nerve tissues/brain tissue, causes pathogenic microorganisms when entering the body are not inactivated and have the available source of Zn/transition metal materials for them to rapidly multiply, and lack chelators with [Zn ions/other transition metal ions]-holding feature to integrate into the Cu ions/Mg ions/Ca ions of the structure of antibacterial alkaline peptides/antibody proteins/microbicidal proteins inside leukocytes, making antibacterial alkaline peptides/antibody proteins/microbicidal proteins inside leukocytes to reduce the ability to kill pathogenic microorganisms, and lack the above chelators to envelope Zn ions/other transition metal ions on the surface of pathogenic microorganisms, weakens the immune system of the elderly, so up to now, there are no pharmaceuticals with the active ingredient being chelators with [Zn ions/other transition metal ions]-holding feature to strengthen the immune system of the elderly in the way that eliminates the above accumulations and in the way that creates the above integrations.

Agents and conditions that increase the lifespan of the host (animal/human), and pharmaceutical situation that increase lifespan:

Until now, due to biology/medicine has not known chelators with [Zn ions/other transition metal ions]-holding feature to help strengthen the immune system of the elderly (as mentioned above), and helps the elderly body clear accumulation of Zn ion-other metal ions complexes in tissues (as mentioned above), contribute to increasing the longevity of the elderly and improving the health of the elderly. Chelators as mentioned above also break Zn crosslinks/other transition metal crosslinks/Zn-other transition metal complex crosslinks in metalloproteins of telomeres during cell division, making the telomeres less likely to stick together at the end of the process of cell division, causing telomeres to shorten slowly, participating in increasing longevity for the elderly (the bodies of long-lived turtles are rich in polyamine derivatives, which helps them have a strong immune system and helps them live a long life and helps them not die when they eat food containing toxins/neurotoxins (such as eating rat poison), their eggs are rich in polyamine derivatives so they do not coagulate when boiled (animal semen of mammals/humans are rich in derivatives of spermidine/spermine, they do not coagulate when heated above 100 degrees Celsius), therefore, up to now, there are no pharmaceuticals with the active ingredient being chelators with [Zn ions/other transition metal ions]-holding feature to help increase the immune system/health/lifespan of the elderly.

Agents and conditions that aggravate Alzheimer's/Parkinson's disease after Alzheimer's/Parkinson's disease patients are exposed to diseases caused by microorganisms, and the condition of pharmaceuticals that prevent Alzheimer's/Parkinson's disease from aggravating by eliminating the agents that aggravate Alzheimer's/Parkinson's disease, and pharmaceutical situation that change conditions that aggravate the disease:

Until now, due to biology/medicine does not know that patients with Alzheimer's/Parkinson's disease have low levels of chelators with [Zn ions/other transition metal ions]-holding feature, so when suffering from these diseases caused by pathogenic microorganisms or eating foods rich in Zn/other transition metals, increases the accumulation of Zn ions/transition metal ions in nerve tissues/brain tissue of these patients, aggravates these diseases, so up to now, there are no pharmaceuticals with the active ingredient being chelators with [Zn ions/other transition metal ions]-holding feature to help avoid aggravation of Alzheimer's/Parkinson's disease in the cases mentioned above in the way that eliminates the accumulation as mentioned above.

Conditions and agents that cause the post-disease syndromes (from diseases caused by pathogenic microorganisms), and the situation of pharmaceuticals to treat these syndromes:

Until now, due to biology/medicine biology/medicine does not know that when hosts suffer from diseases caused by pathogenic microorganisms, their bodies have low levels/depletion of chelators with [Zn ions/other transition metal ions]-holding feature, Zn-rich products/other transition metal-rich products accumulate in metalloproteins of host cells/tissues/nerve tissues/brain tissues, giving rise to post-disease syndrome (such as post-Covid syndrome), so up to now there are no pharmaceuticals with the active ingredient being chelators with strong [Zn ions/other transition metal ions]-holding feature to treat post-disease syndrome (from disease caused by pathogenic microorganisms) in the way as to remove the above accumulations.

The physical structure of diseased tissues (such as infected tissues/necrotic tissues/fungal-infected tissues/tissues containing fluid with many toxins/dark-colored benign tumors/cancerous tumors) plays the role are structures that help protect pathogenic agents/pathogenic entities within these physical structures, and the situation of pharmaceuticals that make these physical structures to lose their ability to protect pathogenic agents/pathogenic entities within these physical structures:

Until now, due to biology/medicine does not know the tight structure/structure containing high viscous fluid of diseased tissues (such as infected tissue/necrotic tissues/fungal-infected tissues/tissues containing high viscous fluid with many toxins/dark-colored benign tumors/cancerous tumors) are structures that protect pathogenic agents/pathogenic entities within these structures, and make the active ingredients to treat tissue diseases very difficult to thoroughly contact pathogenic agents/pathogenic entities within these structures, so up to now, there are no pharmaceuticals that creates strong enough physical effects in diseased tissues to help increase the speed of the active treatment ingredient disperses into the diseased tissue, and create thorough contact between the treatment active ingredient with pathogen agents/pathogenic entities within the treated tissue.

Transition metal ion crosslinks/transition metal ion Zn-other transition metal ion complex crosslinks locate between metal ions in metalloproteins of cancer cells/cancer syncytium/cancerous tissue that are continuously generation, and the situation of pharmaceuticals to treat cancerous tissues in the way that breaks these crosslinks:

Until now, due to biology/medicine does not know that cancer cells/cancer syncytium/cancer tissue that continuously increase in size are transition metal ion crosslinks/Zn ion-other transition metal ion complex crosslinks which located between metal ions in metalloproteins/between metalloproteins of [cancer cells/cancer syncytium/cancerous tissue] causing [cancer cells/cancer syncytium/cancerous tissue] to increase in size continuously; and unknown solution containing [chelators with strong [Zn ions/other transition metal ions]-holding feature+high concentration polyol]/solution containing [chelators with strong [Zn ions/other transition metal ions]-holding feature+high concentration polyol+peroxide (peroxide to create the foam structure for the treated tissue and oxidize the crosslinks mentioned above) when present in cancerous tissue, breaks a large number of the above crosslinks in cancer tissue (makes cancer cells to die/cancer syncytium to disintegrate/cancer tissue to decrease in size), so up to now there are no pharmaceuticals to treat cancer tissues with solution containing [chelators with strong [Zn ions/other transition metal ions]-holding feature+high concentration polyols]/[chelators with strong [Zn ions/other transition metal ions]-holding feature+high concentration polyols+peroxide] for the treatment the cancerous tissues (also for the treatment of benign tumors).

The way in which the structures of external toxins/external neurotoxins attach to host metalloproteins to cause toxicity, and the situation of pharmaceuticals to treat diseases caused by external toxins/external neurotoxins:

Until now, due to biology/medicine does not know the structure of external toxins/external neurotoxins when present in the host body, thy need to hold [Zn ions/other transition metal ions] to attach to metalloproteins of the host, then cause toxicity to the host, so up to now, there are no pharmaceuticals to treat the above structures by separating the above structures from the host's metalloproteins, and by inactivating Zn ions/transition metal ions in the above structures to eliminate the toxicity of external toxins/external neurotoxins, so up to now, there are no pharmaceuticals with the active ingredient being chelators with strong [Zn ions/other transition metal ions]-holding feature to treat diseases caused by external toxins/external neurotoxins to inactivate toxins/neurotoxins.

Agents and conditions that make some pharmaceuticals increase the effectiveness of disease treatment, and situation of chelator pharmaceuticals used together with disease-treatment pharmaceuticals to increase the effectiveness of disease-treatment drugs:

Until now, due to biology/medicine does not know how polyamine derivatives (which are chelators with [Zn ions/other transition metal ions]-holding feature) in the host body that reduce the voltage of the electrode pairs of the host metalloproteins of living entities present in the host body/reduce the polarity of metalloproteins of living entities present in the host body, allowing disease-treatment pharmaceuticals to increase penetration into living entities, increases treatment effectiveness of disease-treatment active ingredients and reduces the doses of disease-treatment active ingredients which need for treatment, so up to now, there are no pharmaceuticals with the active ingredient being chelators with [Zn ions/other transition metal ions]-holding feature) to use with disease-treatment pharmaceuticals to increase the effectiveness of the disease-treatment active ingredient and to reduces the doses of disease-treatment pharmaceuticals that need to use, and there are no pharmaceuticals containing [chelators with [Zn ions/other transition metal ions]-holding feature+active disease-treatment ingredients] to increase the effectiveness of the active disease-treatment ingredients and to reduce the doses of disease-treatment pharmaceuticals that need to use.

Agents and conditions that make bacteria that already have the ability to resist antibiotics increases their ability to resist antibiotics, and the situation of pharmaceuticals that cause bacteria that are already resistant to antibiotics to lose their ability to resist antibiotics:

Until now, due to biology/medicine does not know that chelators with [Zn ions/other transition metal ions]-holding feature are agents that cause microorganisms that already have the ability to resist antibiotics lose this ability, so up to now, there are no pharmaceuticals whose active ingredients are chelators with [Zn ions/other transition metal ions]-holding feature for use as the same time as antibiotics to kill microorganisms, causing the above diseases have lost their ability to resist antibiotics and to help reduce the dose of antibiotics needed to treat diseases caused by pathogenic microorganisms, so far it contains pharmaceuticals with the active ingredient being chelators with [Zn ions/other transition metal ions]-holding feature for use together with antibiotics to cause antibiotic-resistant pathogenic microorganisms to lose their resistance and to help reduce antibiotic doses antibiotics need to be used to treat diseases caused by pathogenic microorganisms, or there are no pharmaceutical in the form of [chelators with [Zn ions/other transition metal ions]-holding feature+antibiotics (function low amount of antibiotic)] to cause antibiotic-resistant pathogenic microorganisms to make the above pathogenic microorganisms to lose their ability to resist antibiotics and to help reduce the doses of antibiotics needed to treat diseases caused by pathogenic microorganisms, so up to now, there are no with the active ingredient being chelators with [Zn ions/other transition metal ions]-holding feature to be used at the same time with antibiotics to make pathogenic microorganisms that are resistant to antibiotics to lose their ability to resist antibiotics, and to help reduce the doses of antibiotics needed to treat diseases caused by pathogenic microorganisms, or do not have pharmaceuticals in the form of [chelators with [Zn ions/other transition metal ions]-holding feature+antibiotics ((low antibiotic content)] to force the antibiotic-resistant pathogenic microorganisms to lose their antibiotic-resistant ability.

Agents and conditions that reduce the voltage measured by the metal-Zn—Cu-metal electrode pair of the patient's body/diseased tissues caused by pathogenic microorganisms/tissues due to mechanical damage, and the situation of pharmaceuticals that reduce the voltage measured above to treat symptoms of pain/inflammation/fever . . . :

Until now, due to biology/medicine does not know the voltage measured by the metal Zn-metal Cu electrode pair of the patient's body fluids/voltage measured on the patient's skin of the patient's body/diseased tissues caused by pathogenic microorganisms/tissues caused by mechanical damage increase dramatically, leading to fever/inflammation/tissue pain/myalgia/joint pain/headache due to the depletion of chelators with [Zn ions/other transition metal ions]-holding feature, so up to now there are no pharmaceuticals containing chelators with [Zn ions/other transition metal ions]-holding feature to treat fever/inflammation/tissue pain/muscle pain/joint pain/headache.

Agents and conditions that cause anaphylactic shock during drug injection/intravenous infusion/blood transfusion/tissue transplantation, and situation of pharmaceuticals to treat anaphylactic shock in the way that eliminates the agents that cause the anaphylactic shock:

Until now, due to biology/medicine has not known that people with very low blood levels of polyamine derivatives, when injected with drugs/vaccines/intravenous infusions/blood transfusions/tissue transplants/contact with foreign allergic substances, these foreign substances entering the body will mobilize chemokines and then chemokines release Zn-rich products, at the same time antibacterial alkaline peptides/antibodies/ceruloplasmins which is rich in Cu but is in a state of lack of integration of polyamine derivatives to the Cu ions in their structure, causing them to become weak Cu chelates (weak Cu holding), instead they are strong Cu chelates (strong Cu holding), causing them to release Cu-rich products. into the host body, the above Zn-rich products and the above Cu-rich products deplete chelators with [Zn ions/other transition metal ions]-holding feature, and under conditions In this depletion, the Cu—Zn complex products become extremely toxic to host cells, Cu—Zn complexes adhere to Fe ions of cells/red blood cells/alveolar cells, cause cells/red blood cells/alveolar cells to lose the ability to exchange O2/produce many neurological symptoms, causing anaphylactic shock, so up to now, there are no pharmaceuticals with the active ingredient being chelators with [Zn ions/other transition metal ions]-holding feature to neutralize the toxicity of the above mentioned Zn—Cu complexes for the treatment the anaphylaxis shock that caused by drug injection/vaccine injection/intravenous infusion/blood transfusion/tissue transplant/foreign allergic substances.

Conditions and agents that prevent microorganisms that cause infectious diseases from spreading, and situation of drugs/products of environmental treatment to prevent the spread of infectious diseases:

Until now, due to biology/medicine does not know that chelators with [Zn ions/other transition metal ions]-holding feature encapsulate [Zn ions/other transition metal ions] in metalloproteins on the surface of pathogenic microorganisms (on the surface of spike proteins of pathogenic viruses, for pathogenic viruses), causing pathogenic microorganisms to lose the ability to spread the disease to other hosts, so up to now, there are no drugs/no products for environmental treatment (sprayed into the environment/wipe on surfaces in environments with infectious diseases) containing the ingredient [chelators with [Zn ions/other transition metal ions]-holding feature+water]/[chelators with [Zn ions/other transition metal ions]-holding feature+propylene glycol/glycerol+water].

The foam structure contains [high concentration polyol+chelator agent 30 low water content] to increase the penetration speed of active ingredients of the pharmaceuticals into diseased tissues with the tight structure/structure containing high viscous fluid, and the situation of pharmaceuticals creating the foam structure containing [high concentration polyol+chelator+low concentration water]:

Until now, due to biology/medicine does not know that when create for diseased tissue the foam structure containing [high concentration polyol+chelator+peroxide+low concentration water], pathogenic entities (such as pathogenic microorganisms/cancer cells)/toxins) in the treated tissue will be located in thin walls containing [high concentration polyol+chelator+peroxide+low concentration water] created by tiny air bubbles, causing pathogenic entities/pathogenic agents quickly die/quickly lose their activity, causing tissue disease (such as infected tissue/necrotic tissue/fungal-infected tissue/tissues tissue containing fluid with many toxins/benign tumors/cancerous tumors) is cured quickly, at the same time, high concentrations of chelators and polyols also help relieve pain/reduce tissue inflammation during treatment, so up to now there are no pharmaceuticals with the ingredients [high concentration polyols+chelator+peroxide+low concentration of water] to treat the above diseased tissues.

Agents and conditions that aggravate many disease types (e.g., cancer/diseases caused by infection of secondary microorganisms in later time/arthritis/many other underlying diseases) in people with post-illness syndrome (such as post-Covid syndrome), and the situation of pharmaceuticals to prevent the aggravation of many disease types as mentioned above:

Until now, due to biology/medicine does not know that after some diseases caused by pathogenic microorganisms (such as Covid 19), especially in people with low levels of polyamine derivatives in blood, Zn ion-other transition metal ion complexes accumulate very persistently in metalloproteins of host cells/tissues/joint tissues/nervous tissues/brain tissues, these accumulations causes many diseases (such as cancer/diseases caused by infection of secondary microorganisms in later time/arthritis/many other underlying diseases) quickly get worse, so up to now, there are no pharmaceuticals with the active ingredient being chelators with [Zn ions/other transition metal ions]-holding feature to clean up the above accumulation to prevent the aggravations of many diseases (such as mentioned above).

The ability to dissolve Zn-blood complexes (or Zn-peptide-complexes) of chelators (this ability indicates the ability to relieve symptoms) and the safety of chelators with [Zn ions/other transition metal ions]-holding feature which are polyamine aminopolycarboxylate/Mg aminopolycarboxylate/Ca aminopolycarboxylate:

Because until now biology/medicine does not know the ability of synthetic chhelators to dissolve Zn-blood complexes (also the ability to relieve symptoms) of [polyamine aminopolycarboxylate/Mg aminopolycarboxylate/Ca aminopolycarboxylate/salts made from polyamines with acids having sulfur functional groups in the molecule/salts made from polyamines having at least 4 nitrogen functional groups in the molecule with acids having at least 2 carboxyl groups in the molecule] have ther higher ability to disolve Zn-blood complex more than this ability of natural chelalators present in the host body (such as lysine phosphate/lysine chloride/arginine phosphate/arginine chloride) is 80 to 200 times, so up to now, there are no pharmaceuticals containing polyamine aminopolycarboxylate/Mg aminopolycarboxylate/Ca aminopolycarboxylat/salts made from polyamine with acid having the sulfur functional group in the molecule/salts made from polymine with at least 4 nitrogen functional groups in the molecule with acids having at least 2 carboxyl groups in the molecule] to treat various diseases by separating [Zn/other transition metal ion]-rich products from host metalloproteins to treat these different diseases and to treat severe cases/critical cases of these different diseases (therefore up to now the treatment of symptoms has been less effective (because contemporary pharmaceuticals cannot eliminate the agents causing the symptoms, just only reduce the perception of pain caused by the symptoms).

The ability to kill bacteria/fungi of several combinations made from chelators with strong [Zn ions/other transition metal ions]-holding feature is far exceeds this ability of antibiotics/fungicides:

Because up to now, due to biology/medicine does not know the ability to kill bacteria/fungi of some combinations made from chelators with strong [Zn ions/other transition metal ions]-holding feature is far exceeds this ability of antibiotics/fungicides, along with the safety of these combinations for the host (host is animal/human), so up to now, there are no pharmaceuticals containing the combinations of chelators to replace antibiotics and fungicides.

For example, US patent application No. US20090030079A1 has the name of the patent application as “Use of trientine and penicilamine as countermeasure to metal contamination” by author Barry, Michael Wells, with the priority date of Jul. 27, 2007, in which methods are provided to minimize/prevent/treat the negative effects of overexposure to metallic contaminants by using trientine or penicillamine or their derivatives, with contaminated metals due to the host being exposed to the source of metals in the environment. The use of trientine or trientine derivatives in US patent application No. US20090030079A1 is unrelated to the pharmaceutical purposes of the patent application related pharmaceuticals containing Zn-holding chelator to treat symptoms/diseases caused by microorganisms/some other diseases . . .

Or for example, U.S. Pat. No. 11,033,579B2 has the name of the patent application as “Use of trientine to deliver copper to ischemic tissue” by author Yujian Jame Kang, with the priority date is Oct. 24, 2015, in which this invention provides a method of using trientine for the purpose of delivering Cu to ischemic heart tissues to increase intracellular Cu levels in heart tissue of patients with heart damage due to ischemic heart tissue. The use of trientine or trientine derivatives in this patent application is unrelated to the pharmaceutical purposes of the patent application related pharmaceuticals containing Zn-holding chelator to treat symptoms/diseases caused by microorganisms/some other diseases . . .

Some inventions use metal chelates to inhibit the proliferation of microorganisms:

For example, US patent application US20070293466A1 titled “Antimicrobial chelates”, authored by Robert Thompson, priority date Nov. 3, 2004, which proposes a method of inhibiting the proliferation of microorganisms. including bacteria and fungi by Zn chelate. While according to the culture experiments of microorganisms decomposing organic matter (which includes bacteria decomposing organic matter with their syncytial in the form of small blocks, and fungi decomposing organic matter with their syncytial in the form of small filaments) during the research process of the invention (of the patent application Pharmaceuticals containing chelators with strong Zn ions-holding feature . . . ) showed that Zn chelates/Fe chelates have almost no ability to inhibit organic matter decomposing microorganisms, Na/K chelates have weak ability to inhibit organic matter decomposing microorganisms, Mg chelates/chelates Ca/Cu chelate has the ability to inhibit microorganisms that decompose organic matter quite strongly, the combination [chelate Cu+chelate Mg/chelate Ca+polyamine derivative] has the ability to inhibit microorganisms that decompose organic matter strongly (far exceeds this inhibition ability of most current antibiotics, and the same goes for the ability to inhibit pathogenic microorganisms on open wounds on infected skin of animals (animals with infected skin used for experiments are marine worms/sipunculus nudus (containing in cans with humidifiers and used as fishing bait) )/on open wounds on infected tissues on human skin. Because pathogenic microorganisms (including pathogenic fungi) need Zn as Zn derivatives to proliferate, therefore only chelators with the ability to hold Zn ions have the ability to inhibit them from proliferating, while Zn chelates do not have this ability, meaning is the proposed method of inhibiting the proliferation of microorganisms including bacteria and fungi by using Zn chelate of the above invention which cannot yield practical results (according to the experiments/theory of the application patent).

Or for example, the Chinese patent application number CN2006100154378A, with the French name “Composition antibioti comprenant des betalacamines et des agents chelateurs ionniques”, by author Hesheng Zhang, which proposes an antibiotic compound consisting of at least at least one β-lactam antibiotic and at least one ionic chelate, and the antibiotic compound comprises at least one β-lactam antibiotic and at least one ionic chelate with the ionic chelate being EDTA disodium, and the compound this antibiotic requires a-lactam antibiotic combined with a chelate ion to create an antibiotic that is more effective than a B-lactam antibiotic. In the patent application number CN2006100154378A, EDTA disodium is used, EDTA disodium is quite toxic to host cells (host is animal/human). Experiments keeping Boraras Micros fish in water with 300 ppm EDTA disodium, showed that the fish died in less than 24 hours; while keeping Boraras Micros fish in water with 800 ppm EDTA Cu/3000 ppm EDTA Mg/12000 ppm EDTA Ca, fish did not die after 10 days. EDTA disodium (as well as EDTA dipotassium) is toxic to host cells due to their non-selective of holding of metal ions due to EDTA disodium holding Mg ions/Ca ions/Cu ions/Fe ions/Zn ions/other heavy metal ions too tightly, whereas metalloproteins of healthy animal/human cells require Mg ions/Ca ions/Cu ions/Fe ions/Zn ions/other heavy metal ions in active form (not be enveloped by chelator types which hold them too tightly) so that the host's healthy cells can function normally.

Or for example, patent application GB0415768.1 with the English name “Treatment of tumor”, by authors Russell Talor and Paul Crees, priority date May 15, 2004, which uses chelating agents (metal ion chelating agents) to produce pharmaceuticals to prevent/treat cancer through intermediaries of bacteria by chelating agent capable of capturing at least one metal ion that the bacteria provide to the cancer which depends on this metal ion to live, in which the chelating agent has the ability to hold Ca ions, and in the implementation example, the chelating agent used is disodium EDTA, while disodium EDTA is highly toxic to healthy cells (as mentioned above), makes the healthy host cells easily are damaged, at the same time, this patent application is not new because the use of sodium calcium EDTA has been used as a food preservative for a long time, and sodium calcium EDTA was introduced for medical use in the United States in 1999. 1953 and is also on the list of essential medicines of the World Health Organization, however sodium calcium EDTA is not included in [pharmaceuticals that directly treat symptoms caused by pathogenic microorganisms/to treat diseases with neurological symptoms caused by pathogenic microorganisms (such as diseases caused by the Sars Covid 2 virus)/pharmaceuticals to directly treat diseases caused by the accumulation of [Zn/Zn-heavy metal complexes] in the host body/in host tissues.

Generally, the prior arts are as follows:

Until now, due to biology/medicine did not know that when the host suffers from diseases caused by pathogenic microorganisms, [Zn derivatives/other transition metal derivatives/other Zn-rich peptides and other transition metal-rich peptides/other Zn-rich proteins and other transition metal-rich proteins] are [Zn/other trasition metal-rich products] transitions] that bind to host metalloproteins, causing symptoms and producing the post-disease syndrome and provide a source of Zn/other transition metal materials for secondary pathogenic microorganisms, and secondary pathogenic microorganisms use high concentrations of [Zn ions/other transition metal ions] on their surfaces to adhere to metalloproteins on the surface of host cells/receptors on the host cell membrane, so up to now, there are no pharmaceuticals containing chelators with [Zn ions/other transition metal ions]-holding feature work by these chelators (of chelator drugs) that separate [Zn/other transition metal]-rich products and separate pathogenic microorganisms from host metalloproteins, work in the way that the chelators separates [Zn/other transition metals]-rich products from host metalloproteins, and separates pathogenic microorganisms from host metalloproteins, and inactivates pathogenic microorganisms which they have not yet bind to host metalloproteins, and clean the accumulation of Zn/transition metal ions in host tissues for the treatment of symptoms in diseases caused by pioneer pathogenic microorganisms and in diseases caused by secondary pathogenic microorganism and to treat all diseases caused by microorganisms and to treat post-disease syndrome.

Up to now, due to biology/medicine does not know that pathogenic microorganisms/metastatic cancer cells have left the cancer tissue/toxins and neurotoxins (including external toxins/external neurotoxins) when present in the host body, their structures hold Zn ions/other transition metal ions in the host body before attaching to the host metalloproteins to cause disease/to metastasize cancer/to toxic, and biology/medicine does not know that these structures are separated from the host metalloproteins and inactivated by chelators with strong [Zn ions/other transition metal ions]-holding feature, so up to now, there are no pharmaceuticals containing chelators with strong [Zn ions/other transition metal ions]-holding feature to treat diseases caused by pathogenic microorganisms/to treat cancer metastasis/to treat diseases caused by toxins and neurotoxins.

Until now, due to biology/medicine does not know that [many physical effects with strong enough/foam structure] when appear in the treated tissue (treated diseased tissue) by the pharmaceuticals containing the agents that generate physical effects with strong enough/foam structure, make the dispersion speed of the active treatment ingredients into the treated tissues to increase by tens to hundreds of times, thereby helping the treatment active ingredients overcome the protection of the dense structure/structure containing high viscous fluid of diseased tissues, which are structures that protect pathogenic agents/pathogenic entities in diseased tissue, so up to now there are no pharmaceuticals containing [treatment active ingredient+agents that generate physical effects with strong enough]/[treatment active ingredient+agents that generate physical effects with strong enough+agent that generate foam structure (such as peroxide)] to treat diseased tissues (such as infected tissue/necrotic tissue/fungal-infected tissue/diseased tissue containing fluid with many toxins/benign tumors/cancerous tumors].

Until now, due to biology/medicine does not know that the weakened immune systems are due to the decline/the depletion of chelators with strong [Zn ions/other transition metal ions]-holding feature in the host body, makes antibacterial alkaline peptides/antibody proteins/microbicidal proteins in leukocytes lack of the amount of these chelators to integrate into Cu ions/Mg ions/Ca ions in their structure, reduces their ability to kill pathogenic microorganisms, causing the host's immune system to weaken its ability to kill pathogenic microorganisms (means to weaken the host's immune system), so up to now, there are no pharmaceuticals containing chelators with strong [Zn ions/other transition metal ions]-holding feature to treat the weakened immune systems caused by chronic diseases/old age.

Up to now, due to biology/medicine does not know that many different treatment pharmaceuticals when combined with chelators with strong [Zn ions/other transition metal ions]-holding feature, the amount of active ingredients of the different treatment pharmaceuticals needed for treatment is very low (but the treatment effectiveness is also increased), so up to now, there are no pharmaceuticals in the form of the combination [active treatment ingredient +chelators with strong [Zn ions/other transition metal ions]-holding feature].

Up to now, due to biology/medicine does not know that pathogenic microorganisms that are resistant to antibiotics will lose this ability when [antibiotics that are resisted by pathogenic microorganisms+chelators with strong [Zn ions/other transition metal ions]-holding feature] are present in the host body, so up to now, there are no pharmaceuticals in the form of the combinations [antibiotics+chelators with strong [Zn ions/other transition metal ions]-holding feature] to treat diseases caused by pathogenic microorganisms that are resistant to antibiotics.

Until now, due to biology/medicine does not know that many strong physical effects with strong enough/foam structures when appearing in the treated tissue (treated diseased tissue) that are generated by the pharmaceuticals contain agents that create physical effects/foam structure, increases the dispersion speed of the active treatment ingredients into the treated tissue by tens to hundreds of times, thereby helping the treatment active ingredients overcome the protection of the dense structure/structure containing high viscous fluid of diseased tissue, which are structures that protect pathogenic agents/pathogenic entities in diseased tissues, so up to now, there are no pharmaceuticals containing [treatment active ingredient+agents that produce physical effects with strong enough]/[active treatment ingredient+a agent that produces physical effects with strong enough+agents that generate foam structure (such as peroxide)] to treat diseased tissues (such as infected tissue/necrotic tissue/fungal infected tissue/diseased tissue containing fluid with many toxins/benign tumors/cancerous tumors].

### BACKGROUND OF INVENTION

In the process of searching for the pharmaceuticals to treat the various diseases mentioned in this invention description, thanks to the implementation of a very large number (more than 400000 experiments) of biological/chemical/physics experiments and clinical trials on a variety of small-size animals (including mammals such as mice) and a series of clinical treatment trials on patients (human) with a number of different diseases (including Covid 19 disease/disease tissues (including severely infected tissues/cancerous tissues), thereby discovering a huge number of elementary foundations in the world of biology, and thanks to these elementary foundations, the invention has found many to treat many different diseases/many different syndromes, especially pharmaceuticals to treat diseases caused by microorganisms of the invention, has the effect of quickly eliminating symptoms, and causing microorganisms to lose their ability to attach to host cells, and quickly weakening pathogenic microorganisms, and strengthening the host's immune system, and prevents the appearance of post-disease syndrome. Invention discovered the to treat diseased tissues (such as infected tissue/necrotic tissue/fungal tissue/benign tumors/cancerous tumors), these pharmaceuticals contain agents that create strong physical effects in treated tissues/create the foam structure for treated tissues, so these pharmaceuticalscause the active ingredients of the pharmaceutical to overcome the tight structure/structure containing highly viscous fluid of the diseased tissues which are the structure protects the pathogenic agents/pathogenic entities inside the diseased tissue, making the speed of dispersal of the active ingredients of the pharmaceutical in the treated tissues faster than that of contemporary pharmaceuticals(which do not produce physical effects/does not produce sufficiently strong physical effects/does not create the foam structure in the treated tissue) from dozens to hundreds of times, thanks to which these pharmaceuticalsof the present invention are effective outstanding treatment of diseased tissues.

Several new elementary foundations which pharmaceutical types of invention based on them for developing, as follows:

In an environment containing water in the body of organisms/host bodies (host is animal/human) such as in blood/tissue fluid/alveolar fluid/intracellular fluid . . . ) in conditions where the body is depleted of chelators with [Zn ions/other transition metal ions]-holding feature (please see these chelator below) the Zn ion forms the complexes with Cu ion/Mg ion/Ca ion/Fe ion/Mn ion/Ni ion/Co ion/Pd ion/Pb ion/Hg ion . . . ], make to generate the complexes [Zn ion-Cu ion/Zn ion-Mg ion/Zn ion-Ca ion/Zn ion-Fe ion/Zn-ion-Mn ion//Zn ion-Ni ion/Zn-ion-Co ion/Zn ion-Pd ion/Zn ion-Pb ion/Zn ion-Hg ion . . . ], the formation of these complexes is similar to the formation of these complexes in electric batteries containing electrode pairs [Zn—Cu/Zn—Mg/Zn—Ca/Zn—Fe/Zn—Mn//Zn—Ni/Zn—Co/Zn—Pd/Zn—Pb/Zn—Hg . . . ] (electrode pairs [Zn-other metals can be in metallic form or in the form of insoluble metal derivatives), and the formation of these complexes generates electric current during the formation of these complexes and changes the redox properties around these complexes during the formation of these complexes. Can recognize the formation of [Zn ion-Cu ion] complex by adding into the test tube containing the solution of [0.1% CuCl2+0.6% ZnCl2 (or greater)] an excess amount of 1% Na ascorbate solution, the resulting is metallic Cu suspension will not appear; while metallic Cu suspension will appear when add in the test tube containing 0.1% CuCl2 of an excess amount of 1% Na ascorbate solution, the resulting is metallic Cu suspension will appear; and metallic Cu suspension will not appear when add in the test tube containing the solution of [0.1% CuCl2+ several percent [arginine/lysine/triethylenetetramine/triethylenetetramine glutamate/ triethylenetetramine diethylenetriaminepentaacetate/Mg EDTA/Mg DTPA] an excess amount of 1% Na ascorbate solution.

The biological significance of the discoveries mentioned in the above section (discovery of the formation/existence of [Zn ion-other metal ions] complexes in aqueous environments) has a very wide scope, in which such as:—when the host suffers from diseases caused by pathogenic microorganisms/some other diseases, the chelators are depleted (also known as chelating agents, and these chelators are mainly polyamines derivatives such as derivatives of [histidine/lysine/arginine/agmatine/cadaverine/putrescine/spermidine/spermine . . . ], these polyamine derivatives are chelators with [Zn ions/other heavy metal ions]-holding feature), this depletion is due to these chelators fully held Zn derivatives released from pathogenic microorganisms and from fragments of pathogenic microorganisms, and because these chelators fully held [Fe ions/Cu ions/Zn ions/other heavy metal ions] from host cell/host red blood cell fragments (attacked by pathogenic microorganisms);—in conditions where the host body is depleted of these chelators, the Zn ions of these Zn derivatives will attach to the metal ions in the structure of peptides (soluble)/proteins (soluble) in the blood/alveolar fluid/tissue fluid, generates Zn-peptide complex suspension/Zn-protein complex suspension, causing symptoms of thick blood/thick tissue fluid/thick alveolar fluid;—in conditions of the host body is depleted of these chelators, pathogenic microorganisms will easily accumulate Zn ions/other heavy metal ions to meet their needs for rapid multiplication, leading to the disease condition of the pioneer disease and secondary diseases caused by pathogenic microorganisms to quickly worsen; “adhering-Zn ion pins” in proteins on the surface of pathogenic microorganisms (on spike proteins, for viruses) at free form (not enveloped by chelators with [Zn ions/other transition metal ions]-holding feature), so they easily adhere to metal ions in metalloproteins on the surface of the host cell to attack the host cells/to enter into the host cell; −Zn ions of Zn derivatives will attach to metal ions in the structure of metalloproteins of host cells/host tissues, giving rise to various symptoms/various neurological symptoms/blood clots/cytokine storm/respiratory failure/acute respiratory failure] (collectively referred to as symptoms);—when above chelators are not depleted and have high enough concentrations in the host blood, they will hold [Zn ions/other metal ions], helping to reduce the accumulation of [Zn ions/other heavy metal ions/Zn ion-other heavy metal complexes] in [blood/tissue fluid/alveolar fluid/tissue/nerve tissue/brain tissue], thereby preventing/relieving symptoms and preventing/relieving syndrome due to the accumulation of Zn ions/other heavy metal ions/Zn ion-other heavy metal complexes] in [tissues/nervous tissues/brain tissue] that causes post-disease syndromes (such as post-Covid syndrome/brain fog syndrome in people who have had Covid 19).

In an environment containing water in the body of organisms/host bodies (host is animal/human) such as in blood/tissue fluid/alveolar fluid/intracellular fluid . . . ) in conditions where the body is depleted of chelators with [Zn ions/other transition metal ions]-holding (such as the amount of chelators with [Zn ions/other transition metal ions]-holding remains is not significant in the patient's body), Cu ions will attach to [Fe ions in metalloproteins of [host cells/red blood cells/alveolar cells], causing these cells to lose the ability to exchange O2, causing symptoms of acute respiratory failure (not due to lung infection), and this symptom is very dangerous because it can quickly lead to death. Among the bond forces between Cu ions and other metal ions, the bond force of the Cu ion-Fe ion complex is the strongest, can recognize the existence of Cu ion-ion Fe ion complex/Cu ion-Zn ion complex/Cu ion-Mg ion complex/Cu ion-Ca ion complex, and can recognize Cu ion-other metal ion bonds, in which the Cu ion-Fe ion bond is the bond type with the highest magnitude through experiments conducted in water environment (water environment without chelators with [Zn ions/other transition metal ions]-holding) is the environment that simulates the blood environment/alveolar fluid environment of a host with the depletion of these chelators), these experiments were performed by adding an excess amount of 1% Na ascorbate solution to the test tube containing the solution with [0.1 % CuCl2+greater than 0.2% FeCl3]/the test tube containing the solution containing [0.1 % CuCl2+greater than 0.6% ZnCl2]/the test tube containing the solution containing [0.1 % CuCl2+greater than 0.63% MgCl2]/the test tube containing the solution conatining [0 . 1 % CuCl2+greater than 0.65% CaCl2], results are metallic Cu suspension will not appear in these test tubes. Experiments help identify that when the depletion of chelators with [Zn ion/Cu ion/other transition metal ions]-holding in the patient's body, the patient will suffer acute respiratory failure and will easily lead to death, these experiments were performed by keeping Boraras Micros fish in water containing 0.4 ppm CuCl2, resulting in the fish dying after a few hours;-keep Boraras Micros fish in water containing [0.4 ppm CuCl2+a few ppm to more than 10 ppm of [lysine phosphate/arginine phosphate/Ethylenediaminetetraacetic acid magnesium (Mg EDTA)/Diethylenetriaminepentaacetic acid magnesium (Mg ethylenediaminetetraacetate, Mg DTPA)/Ethylenediaminetetraacetic acid calcium (Ca ethylenediaminetetraacetate, Ca EDTA)/Diethylenetriaminepentaacetic acid calcium (Ca diethylenetriaminepentaacetate, Ca DTPA)/triethylenetetramine diethylenetriaminepentaacetate], the fish will not die after several weeks.

Experiments help identify the active ingredients used as ingredients of invention chelator pharmaceuticalsthat are not toxic to host cells (host is animal/human) at the concentration in host blood necessary for treatment the diseases (depending on the type, this concentration in the host's blood is needed for the disease treatment, in which the safe concentration threshold is quite high for polyamine aminopolycarboxylate/Mg aminopolycarboxylate, the safe concentration threshold is very high for Ca aminopolycarboxylate, average safety level for Cu aminopolycarboxylate (however, when Cu aminopolycarboxylate is combined with other chelators, the safety threshold of Cu aminopolycarboxylate will be raised quite high). These safety thresholds can be identified through the experiments are as follows:—keeping Boraras Micros fish in water containing [800 ppm EDTA Cu/3000 ppm EDTA Mg/12000 ppm EDTA Ca], the result is fish will not die after 10 days;—keep Boraras Micros fish in water containing [800 ppm EDTA Na, the result fish will die within 24 hours (because EDTA Na does not selectively hold metal ions, including metal ions that help exchange O2 in the fish's gill such as Fe ions, which will cause acute respiratory failure in fish, while polyamine derivatives/EDTA Cu/EDTA Mg/EDTA Ca will hold Fe ions weakly (not too tightly so it will not cause acute respiratory failure in fish). The biological significance of these discoveries is: −Cu chelates/Mg chelates/Ca chelates are not toxic to animal cells (the bodies of molluscs/arthropods are rich in Cu chelates/Cu ligands that are Cu-histidine complexes, and humans also usualy eat foods rich in Cu chelates/Cu ligands from molluscs/arthropods);—the depletion of derivatives with Cu ions holding feature in the body of patients with diseases caused by pathogenic microorganisms, will cause acute respiratory failure symptoms as mentioned above, and this symptom is very dangerous and can easily lead to quick death;-urgently introduce into the body of patients with acute respiratory failure symptoms in diseases caused by pathogenic microorganisms (especially Covid 19 disease) the invention pharmaceuticals contain chelators with [Cu ions/Zn ions/other heavy metal ions] holding feature (referred to as chelator drugs) (such as EDTA Mg/DTPA Mg/EDTA Ca/DTPA Ca/triethylenetetramine diethylenetriaminepentaacetate) to promptly save patients from death by acute respiratory failure symptoms.

Pathogenic microorganisms are living entities that grow rapidly (such as organic matter decomposing microorganisms/pathogenic microorganisms/pathogenic microorganism syncytial/cancer cells/cancer syncytial/cancerous tissues . . . ), so they have a high demand for Zn and other transition metals, the proteins of pathogenic microorganisms are rich in cysteine, which helps these proteins accumulate Zn ions/other transition metal ions, making their proteins (especially the proteins on their surfaces) are rich in Zn/other transition metals). When the host (animal/human) suffers from diseases caused by pathogenic microorganisms, there will be 3 stages related to the following symptoms:—The first stage is the pathogenic microorganisms stage and their fragments release [Zn derivatives/other transition metal derivatives/Zn-other heavy metal complex derivatives/Zn-other heavy metals-rich peptides and proteins (referred to as are [Zn/other transition metals]-rich products from pathogenic microorganisms, and [Zn/other transition metal]-rich products from pathogenic microorganisms that are also toxins/neurotoxins) into [host blood/tissue fluid/alveolar fluid], at this stage, chelators with [Zn/other transition metals] holding feature in the host body are still abundant (not including people with weakened immune systems/the elderly), so [Zn/other transition metals]-rich products present in the host body holding [Zn ions/other heavy metal ions] of [Zn/other transition metals]-rich products from pathogenic microorganisms (natural chelators present in the host body are polyamine derivatives, such as derivatives of histidine/lysine/arginine/cadaverine/putrescine/spermidine/spermine . . . ), makes [Zn/other transition metals]-rich products from pathogenic microorganisms cannot adhere to the host's metalloproteins, so during this stage symptoms rarely appear, and this stage is the stage when the amount of chelators with [Zn/other transition metals]-holding feature that retains the ability of holding of Zn ion/other transition metal ions] in the patient's body decreases sharply, and leads to the period when the patient's body is depleted of active chelators with [Zn/other transition metals] holding feature. The second stage is the stage in which the above-mentioned chelators in [blood/tissue fluid/alveolar fluid] are depleted due to the holding of [Zn ions/other transition metal ions] of Zn-rich products/other transition metals-rich products from pathogenic microorganisms, and under conditions of depletion of these chelators, Zn-rich products/other transition metals-rich products from pathogenic microorganisms and pathogenic microorganisms easily attaches to the host's metalloproteins, causing symptoms/toxicity to host cells and host tissues, host nervous tissue, and host brain tissue, causing disease in the host and causing post-disease syndrome for the host (in some diseases, such as post-Covid syndrome). Products rich in [Zn/other transition metals] from pathogenic microorganisms, when attached to host metalloproteins, generate various symptoms (such as fatigue/fever/rash/drainage nose/red eyes/diarrhea/mucus thick . . . )/various neurological symptoms (such as loss of taste/loss of smell/loss of appetite/sore throat/muscle pain/joint pain/headache. . . )/blood clots/high viscosity of [blood/tissue fluid/alveolar fluid]/cytokine storm/respiratory distress] (collectively referred to as symptoms), and when some symptoms are more intense will cause the host to die; besides, the abundance of [Zn/other transition metals]-rich products in the patient's body is also a rich source of [Zn/other heavy metals] for secondary microorganisms to use to multiply rapidly, causing secondary diseases to quickly become worse. In addition, the abundance of Zn derivatives/Zn-other heavy metal complex derivatives as mentioned above also causes the accumulation of Zn ion/Zn ion-other heavy metal ion complexes in tissues/nerve tissues/brain tissue, giving rise to post-disease syndrome (such as post-Covid syndrome in people who have had Covid disease and their bodies go through a long period of depletion of the chelators as mentioned above).

Experiments of introduction dilute Zn derivatives into contact with open wounds on animal skin (the animals used were marine worms/sipunculus nudus (marine animals kept in boxes with materials kept moist with sea water to used as fishing bait, these animals have the ability to live in the air for long periods of time in environments containing)/mammals, resulting in infections on open wounds increasing rapidly, and these infected open wounds are almost impossible to cure by antibiotics, but can only be cured with chelators with [Zn/other heavy metals]-holding feature. The significance of these experiments is to help identify that when patients suffer from diseases caused by pioneering microorganisms (such as the Sars Covid 2 virus that causes Covid 19), Zn derivatives/Zn-rich peptides are released from Sars Cov 2 and from fragments of Sars Cov 2 is the source of Zn that makes secondary microorganisms to cause infections (secondary pathogenic microorganisms) to multiply strongly, causing secondary diseases (such as lung infections/fungal infections) that rapidly worsen, and help identify secondary diseases accompanying pioneering diseases such as Covid 19 due to increased concentrations of Zn derivatives/Zn-rich peptides in the patient's blood/tissue fluid/alveolar fluid, making the treatment of secondary diseases difficult. This outbreak (such as severe lung fungal infections/infections) is very difficult to cure, and requires treatment with the invention chelator pharmaceuticalsto recover quickly.

Experiments of cultivating microorganisms that decompose organic matter in the controlled liquid mediums which does not contain substances that promote the growth of microorganisms that decompose organic matter (such as no addition of [ZnCl2/FeCl3/CuCl2/citrate Na/succinate Na/malate Na/gallate Na/tannate Na/Boron]), and experiments on cultivating microorganisms that decompose organic matter in the liquid mediums which are supplemented with each substance is [ZnCl2/FeCl3/CuCl2/Na citrate/Na succinate/Na malate/Na gallate/Na tannate Na] with different concentrations, resulting in microorganisms that decompose organic matter (including bacteria/fungi) multiply strongly when the above culture mediums having [12 ppm ZnCl2/[6 ppm ZnCl2+2 ppm CuCl2+12 ppm FeCl3]/80 ppm citrate Na/80 ppm succinate Na/80 ppm malate Na/5 ppm Na gallate/30 ppm tannate]. And when added to the above environments Cu EDTA/Mg EDTA/Ca EDTA/triethylenetetramine diethylenetriaminepentaaceate at the same concentration of 150 ppm/400 ppm/700 ppm/100 ppm, then the proliferation of microorganisms that decompose organic matter will be strongly inhibited, while adding Bactrim/Ampicillin to the above environments with concentrations of 20 ppm/10 ppm, respectively, then the proliferation of microorganisms that decompose organic matter will be just only slightly inhibited in the first 36 hours. The significance of these experiments is to help identify substances that promote the proliferation of microorganisms (including pathogenic microorganisms already present in the patient's body,, thanks to experiments testing these substances on open wounds on the animal skin), and when patients have diseases caused by pathogenic microorganisms, it is necessary to limit foods/drinks/supplements which are rich in [Zn/citrate/succinate/malate/gallate/tannate], especially do not use Zn supplement tablets during the disease to avoid aggravating secondary diseases as well as avoiding aggravating symptoms that can lead to death if high doses of Zn supplements are used, and help identify Mg aminopolycarboxylate/Ca aminopolycarboxylate/polyamine aminopolycarboxylate as good raw materials to make chelator pharmaceuticalsto treat diseases caused by pathogenic microorganisms, and these pharmaceuticalshelp partially replace the antibiotics/help completely replace the antibiotics (completely replace the antibiotics (complete replace the antibiotics (completedly replace the antibiotics as for polyamine aminopolycarboxylate salts made from Triethylenetetramine (TETA)/Tetraethylenepentamine (TEPA)/Pentaethylenehexamine (PEHA)/Branched Poly(ethyleneimine) (branched PEI) with diethylenetriaminepaentaacetic acid (DTPA), or as for salts made from branched TETA/TEPA/PEHA/PEI with sulfur functional acids in the molecule, or as for chelator combinations containing Cu aminopolycarboxylate).

Under conditions of depletion of chelators with [Zn ions/other heavy metal ions]-holding feature, the suspension composed of Zn-blood-complexes will appear in the blood, with very low concentrations of Zn derivatives (below 0.7-1.5 ppm calculated as Zn) is enough to form the Zn-blood-complex suspension, and chelators with [Zn ions/other heavy metal ions]-holding feature are capable of dissolving the Zn-blood-complex suspension (at neutral to alkaline pH). Can recognize the Zn-blood complex suspension are generated by experiments:—Gradually add into the containing test tube the solution contains 0.01% blood (this 0.01% blood solution is mixed from water and sample blood of [animal/human] with sample blood containing 6% NaCl to help prevent the sample blood from coagulating before being used for experiments, the purpose of using this solution contains a very small amount of blood is to avoid polyamine derivatives present in the blood affecting the experimental results because the blood of healthy hosts is rich in polyamine derivatives) and 0.001 % ZnCl2 and shake well, the result is when the ZnCl2 concentration is above 1.5 ppm, the Zn-blood suspension begins to appear, and this suspension appears abundantly when the ZnCl2 concentration reaches the concentration of 2 ppm; gradually add into the test tube containing Zn-blood complex suspension with the concentration of 2 ppm ZnCl2 solution [0.1% arginine phosphate/0.1% arginine glutamate/0.01% EDTA Mg/ 0.01% triethylenetetramine gluconate/0.01% triethylenetetramine diethylenetriaminepentaacetate] until the suspension is completely dissolved and the solution becomes clear, this test shows the amount of [EDTA Mg/triethylenetetramine gluconate/triethylenetetramine diethylenetriaminepentaacetate/tetraethylenepentamine diethylenetriaminepentaacetate/pentaethylenehexamine diethylenetriaminepentaacetate/branched polyethyleneimine diethylenetriaminepentaacetate] (which are synthetic polyamine derivatives) needed to completely dissolve the Zn-blood complex suspension is tens to hundreds of times lower than the amount of lysine phosphate/lysine chloride/arginine phosphate/arginine chloride (which are natural polyamine derivatives). The significance of these examinations is when the host suffers from diseases caused by pathogenic microorganisms and reaches the stage where the host's body is depleted of chelators with [Zn ions/other heavy metal ions]-holding feature, then a very low concentration of [Zn derivatives released from pathogenic microorganisms and fragments of pathogenic microorganisms] in the blood (just above 1.0 ppm calculated as Zn) is enough to create Zn-blood complex suspension, which means that when the above-mentioned chelators are depleted and the concentration of Zn derivatives from pathogenic microorganisms increases, a series of symptoms will be generated even when the concentration of Zn derivatives is increased in the host's blood is very low, and when symptoms have appeared with great intensity, the introduction into the host body of small amounts of chelators with [Zn ions/other heavy metal ions]-holding feature, the Zn-blood complex suspension will dissolve and most symptoms will rapidly disappear. The above experiments also help to know the effects including the effect of the eliminating of symptoms/the effect of helping the host body of recovering/the effect of preventing the generation of syndrome of chelators (such as the active ingredients mentioned above) of the invention chelators (because the active ingredient are chelators, therefore they briefly are called chelator drugs) is far exceeds the effects of natural chelators that present in the host body (host id animal/human). Clinical trials of treating Covid 19 with the invention pharmaceutical containing [1.4 g EDTA Mg/0.8 g triethylenetetramine diethylenetriaminepentaacetate], showed that most symptoms improved significantly after 4 hours after taking the first dose, and after 48 hours the remaining symptoms were insignificant and after a few days the patient was cured, and after the treatment period (including 7 days of treatment, and 8 days of taking ½ treatment dose to prevent post-Covid syndrome), the post-Covid syndrome does not appear.

The active ingredient polyamine aminopolycarboxylate, such as polyamine aminopolycarboxylate is made from polyamine which polyamine is Triethylenetetramine/ Tetraethylenepentamine/Pentaethylenehexamine/Polyethylenimine branched with aminopolycarboxylic acid is Ethylenediaminetetraacetic acid (EDTA)/Diethylenetriaminepentaacetic acid (DTPA)/triethylenetetramine-N, N, N′, N″, N′″, N′″-hexaacetic acid (TTHA)/Dibenzothiophene-1,3,6,8-Tetracarboxylic acid (DTCA)/ethylene glycol bis(2-aminoethyl ether)-N, N, N′, N′-tetraacetic acid (EGTA)/1,2-bis(2-aminophenoxy)ethane-N, N, N′, N′-tetra-acetic acid (BATPA)/1,4,7-triazacyclononane-1,4,7-triacetic acid (NOTA)/1,4,7,10-tetraazacyclododecane-1,4,7,10-tetraacetic acid (DOTA)/N, N′-bis(2-hydroxyethyl)ethylenediamine-N, N′-diacetic acid (HEEDA)/1,4,7,10,13-pentaazocyclopentadecane pentaacetic acid (PEPA)/1,4,7,10,13,16-hexaazocyclooctadecane hexaacetic acid (HEHA)/1,4,8,11-tetraazacyclotetradecane-1,4,8,11-tetraace-tic acid (TETA)/triethylenetetraminepentaacetic acid (TETPA)/4,5-diphenyl-1,3-oxazol-2-yl)thio](phenyl)acetic acid (DOTPA)/1,4,7,10-tetraaxacyclododecane-1,4,7,10-tetramethylenephosphonic acid (DOTMP)/Ethylenediamine-N, N′-disuccinic acid (EDDS)/2-hydroxyethyliminodiacetic acid (HEIDA)/Glutamic acid diacetate (GLDA)/Methylglycinediacetic acid (MGDA)/1-aspartic acid N, N-diacetic acid (ASDA)/1,2-cyclohexylenediamine tetra-acetic acid (CDTA)/ethylenediamine-N, N′-bis(2-hydroxyphenylacetic acid) (EDDHA)/ethylene glycol bis(2-aminoethyl ether)-N, N, N′, N′-tetraacetic acid (EGTA)/[2-{4,7-biscarboxymethyl(1,4,7)triazacyclonona-1-yl-ethyl} carbonylmethylamino] acetic acid (NETA)/1,2-cyclohexylenedinitrilotetraacetic acid (CyDTA)/triethylenetetraaminehexaacetic acid (TTHA)/1,4,7,10-Tetraaza-cyclododecane-1,4,7-triacetato (DO3A)/6-amino-6-methylperhydro-1,4-diazepinetetraacetic acid (AAZTA) are chelators that neutralize the toxicity of toxins/neurotoxins (including toxins/neurotoxins produced by pathogenic microorganisms in the patient's body and external toxins/external neurotoxins (such as from rat poison/pesticides/venom of venomous animals/poisonous plants/poisonous mushrooms/toxins produced from technology) biological/some other organic and inorganic toxins), the active ingredient triethylentetramine diethylenetriaminepentaacetate and some other polyamine aminopolycarboxylates as mentioned above have been used for clinical treatment of small animals (such as Boraras Micros fish/flies and fly larvea/cricket/earthworm/seaworms/sipunculus nudus/chicken/mouse which they are contaminated with toxins/neurotoxins that are the active ingredients of many rat poisons/many pesticides/some poisonous plants . . . , and all have achieved results that help experimental animals not die despite being exposed to doses of toxins/neurotoxins many times to dozens of times of the minimum amount of these toxins/neurotoxins that makes them to die.

Recognizing the ability to neutralize the toxicity of toxins by the active ingredient that is triethylentetramine diethylenetriaminepentaacetate through clinical trials on Boraras Micros fish as follows:
keeping fish in water with [5 ppm Nereistoxin+50-300 ppm triethylenetetramine diethylenetriaminepentaacetate], fish lived for many days (up to weeks), while keeping fish in water with 5 ppm Nereistoxin fish will die after 30 minutes;
keep fish in water containing [1.25 ppm flocoumafen (¼ tablet of rat poison (with 0.005% flocoumafen) in 1 liter of water)+50-300 ppm triethylenetetramine diethylenetriaminepentaacetate], fish lived for many days (up to weeks), while keeping fish in water containing 1.25 ppm flocoumafen, fish will died after 36 hours;
keeping fish in water with [0.3 ppm Bromadiolone+50-300 ppm triethylenetetramine diethylenetriaminepentaacetate], fish lived for many days, while keeping fish in water with 0.3 ppm Bromadiolone, fish died within 12 hours;
keeping fish in water with [0.2 ppm Methylamine Avermectin+50-300 ppm triethylenetetramine diethylenetriaminepentaacetate], fish lived for many days, while keeping fish in water with 0.2 ppm Methylamine Avermectin, fish died within 12 hours;
keeping fish in water with [2.5 ppm Emamectin benzoate+50-300 ppm triethylenetetramine diethylenetriaminepentaacetate], fish lived for many days, while keeping fish in water with 2.5 ppm Emamectin benzoate, fish died within hours;
keeping fish in water with [1.0 ppm phenylperazole+50-300 ppm triethylenetetramine diethylenetriaminepentaacetate], fish lived for many days, while keeping fish in water with 1.0 ppm phenylperazole, fish died within a few hours;
keeping fish in water with [[0.2 ppm Alpha Cypermethrin+50-300 ppm triethylenetetramine diethylenetriaminepentaacetate], fish lived for many days, while keeping fish in water with 0.2 ppm Alpha Cypermethrin, fish died within a few hours;
keeping fish in water with [0.3 ppm CuCl2+20 ppm ZnCl2+20 ppm FeCl3], the fish lived for many days, while keeping fish in water with [0.3 ppm CuCl2+20 ppm ZnCl2], the fish died in a few hours; these experiments is to help identify that during the hemorrhagic stage of dengue fever, the patient's fever reduces and feels comfortable due to the increased concentration of Fe derivatives in the blood (from broken red blood cells);
keep fish in water with 2000 ppm triethylenetetramine diethylnenepentaacetate/3000 ppm Mg EDTA/2000 ppm Cu EDTA/12000 ppm Ca EDTA/[10000 ppm+2500 ppm CuCl2], fish lived for many days, while keeping fish in water with 1000 ppm Na2 EDTA/0.5 ppm CuCl2/[0.3 ppm CuCl2+20 ppm ZnCl2], fish died within a few hours. These experiments help identify the toxicity threshold of above chelators on host cells (host is animal/human), and Ca EDTA/Ca DTPA are highly safe and help treat poisoning with heavy metal derivatives/radioactive metal derivatives with the contamination amounts that are extremely high.

Recognizing the ability of the active ingredient that is triethylenetetramine diethylenetriaminepentaacetate to inhibit/kill pathogenic microorganisms by clinical experiments on Boraras Micros fish as follows: use a quantity of Boraras Miros fish that died after 3 days (about 100 g), then put this amount of dead fish into 10 water liters and keep the fish for 2 days so that all fish in this 10 water liters are infected with pathogenic microorganisms from 100g of fish died after 3 days, then add 1 water liter containing the above dead fish into each large beaker and each beaker contains about 30 live fishes; then use 1 beaker as a control experiment, the remaining beakers add substances so that each cup contains 10 ppm Ampicillin/20 ppm Bactrim/20 ppm Ampicillin/40 ppm Bactrim/100 ppm triethylenetetramine diethylenetriaminepentaacetate/300 Mg EDTA/600 ppm Ca EDTA/50 ppm Cu ETDA+100 triethylenetetramine diethylenetriaminepentaacetate/[0.3 ppm Ampicillin+50 ppm triethylenetetramine diethylenetriaminepentaacetate]/[0.6 ppm Bactrim+50 ppm triethylenetetramine diethylenetriaminepentaacetate]; the results are after 6 days, fishes in the control experimental beaker/beakers containing [10 ppm Ampicillin/20 ppm Bactrim/20 ppm Ampicillin/40 ppm Bactrim] all of fishes died and the water in these beakers became cloudy; and after 6 days fish in beakers with [100 ppm triethylenetetramine diethylenetriaminepentaacetate/ 300 Mg EDTA/600 ppm Ca EDTA/50 ppm Cu ETDA+100 triethylenetetramine diethylenetriaminepentaacetate/[0.3 ppm Ampicillin+50 ppm triethylenetetramine diethylenetriaminepentaacetate]/[0.6 ppm Bactrim+50 ppm triethylenetetramine diethylenetriaminepentaacetate], the fishes did not die and the water in these cups was transparent.

The above results also indicate polyamine aminopolycarboxylate when have the concentration in [blood/tissue fluid/alveolar fluid] of the host are quite low (over 30 ppm for polyamine aminopolycarboxylate made from TEPA/PEHA/branced PEI with DTPA), and above 60 ppm for polyamine aminopolycarboxylate formed from TETA with DTPA), the ability to inhibit/kill pathogenic bacteria far exceeds this ability of Bactrim with the concentration of 10 ppm/Ampicillin with the concentration of 20 ppm in host [blood/tissue fluid/alveolar fluid].

In particular, pharmaceuticals with the ingredients [polyamine aminopolycarboxylate+antibiotic active ingredient]/[polyamine aminopolycarboxylate+salt made from polyamine with at least one acid with the sulfur functional group in molecule] are pharmaceuticals that kill pathogenic microorganisms very strongly, Therefore, they are outstandingly effective pharmaceuticals in treating diseases caused by pathogenic microorganisms, and helps to greatly reduce the doses of active ingredients of antibiotics/not having to use antibiotics, while also bringing a series of other benefits as mentioned above (such as quickly reducing symptoms/cleansing the body from the accumulation of [Zn ion accumulation/other transition metal ions]/relieving pain and inflammation . . . ).

Experiments help identify Zn-rich derivatives (simulating Zn-rich products from pathogenic microorganisms) as agents that generate the symptoms of loss of taste and various symptoms (such as in Covid 19/influenza/diseases caused by pathogenic microorganisms):—the symptom of loss of taste caused by Zn derivatives can be identified by chewing and sucking a 15 mg Zn gluconate tablet and spitting it out after 120 seconds, then tasting the solution [15% sucrose/aspartame sweet enough/sucralose sweet enough], the sweet taste sensation on the tongue is completely lost; then suck the solution [5% arginine glutamate/0.5% triethylenetetramine glutamate] for 10 seconds and taste the solution again [15% sucrose/aspartame sweet enough/sucralose sweet enough], resulting in the sweet taste on the tongue are fully recovered.

Experiments help identify Zn-rich derivatives (simulating Zn-rich products from pathogenic microorganisms) as agents that generate symptoms (such as itchy throat/sore throat/cough/runny nose/body aches/slight fever/headache . . . are similar to these symptoms in diseases caused by pathogenic microorganisms (such as flu/viral fever/Covid 19 . . . ) , through experiments (this experiment is only for experts because it will cause uncomfortable symptoms for many hours to more than 24 hours, and triethylenetetramine derivatives should be available to take orally to quickly relieve these symptoms) by sucking 2 ml of 0.4% ZnCl2 solution (which contains about 80 mg of ZnCl2) for 120 seconds and let a little of this solution pass through the throat and then spray out, resulting in above symptoms appear after a few minutes to a few hours; then slowly drink a few ml of solution [10% triethylenetetramine glutamate/10% triethylenetetramine ascorbate], resulting in the above symptoms will gradually disappear after a few hours, and the headache symptom will disappear last.

Experiments help identify the agents that causes symptoms of increased blood viscosity/symptoms of blood clots/symptoms of bleeding in dengue diseases through the results of the following experiments:—add 1.4 ml of 0.1% ZnCl2 solution into the test tube containing 100 ml of 0.9% NaCl solution and containing 1% blood (mammalian blood/human blood, and this blood is added with NaCl in order the NaCl having 6% to prevent the blood from clotting before it is diluted with water), the result is the suspension with Zn-blood complex component begins to appear (and when add more 0.1% ZnCl2 solution into the test tube, the suspension density will correspondingly increase);—add 6 ml of 0.1% FeCl3 solution into the test tube with the above suspension, the result is the suspension will completely dissolve and the solution becomes transparent;—add 4 ml of neutral [NaH2PO4+Na2HPO 4] solution to the transparent solution, the result is the suspension to reappear;—repeat the first experiment to have the test tube containing the suspension with Zn-blood complex component, and then add into this test tube 2 ml of 10% neutral arginine phosphate solution (3 parts arginine: 1 part H3P 4 (type 85% H3PO4) ), the result is the suspension completely dissolves and the solution becomes transparent;-repeat the first experiment to have the test tube containing the suspension with Zn-blood complex component, and then add 1.0 ml of 10% neutral arginine glutamate solution (1 part glutamic acid: 1, 36 parts of arginine), the result is the suspension completely dissolves and the solution becomes transparent;—repeat the first experiment to have the test tube containing the suspension with Zn-blood complex component, and then add 0.7 ml of arginine ascorbate (1 part arginine:1 part ascorbic acid) to this test tube, ), the result is the suspension completely dissolves and the solution becomes transparent;—repeat the first experiment to have the test tube containing the suspension with Zn-blood complex component, and then add 2 ml of 0.1% EDTA Mg solution to this test tube, the result is the suspension completely dissolves and the solution becomes transparent;—repeat the first experiment to have the test tube containing the suspension with Zn-blood complex component, and then add 1.2 ml of triethylenetetramine diethylenetriaminepentaacetate into this test tube, the result is the suspension completely dissolves and the solution becomes transparent. The above results and the results of other experiments show the ability to dissolve the suspension with Zn-blood complex component of chelators used as active ingredients of the present invention chelator pharmaceuticals such as Mg EDTA/Ca EDTA/Mg DTPA/Ca DTPA/[salts formed from TETA/TEPA/PEHA/PEI branched with DTPA] is powerful than lysine phosphate/lysine chloride/arginine phosphate/arginine chloride from 70 times to more than 200 times, these help identify that the present invention pharmaceuticals invention have the ability to [relieve symptoms/weaken pathogenic microorganisms/strengthen the immune system] far exceeds this ability of natural chelators (mainly natural polyamine derivatives) present in the host body, thanks to which the present invention pharmaceuticals have the ability to help save the lives of severe cases/critical cases of diseases caused by pathogenic microorganisms include severe cases/critical cases of dangerous diseases with high/very high mortality rates (such as Covid 19/avian influenza).

Experiments of measuring the voltage obtained from a pair of electrodes [metallic Zn-metallic Cu] (with the surface of each electrode having a fabric layer holding gel solution+polarizer (such as NaCl)) applied to the skin surrounding [inflamed tissue/infected tissue], and the control measuring is this electrode pair to apply to the skin of healthy tissue, resulting in the voltage obtained from [inflamed tissue/infected tissue]] being much higher than the voltage obtained from healthy tissue, the voltage obtained from [inflamed tissue/infected tissue] is high because [inflamed tissue/infected tissue] depleted of chelators with [Zn ions/Cu ions/Fe ions/metal ions other heavy] holding feature. In the metalloproteins of [tissue/nerve tissue/brain tissue] there are [Zn-Cu/Zn-other metals/Cu-other metals] electrode pairs (these electrode pairs are in the form of insoluble metal derivatives), under the condition of depletion of the above chelators in the above tissues (as well as in the host body), the voltage of the above electrode pairs will increase, and this increasing will cause symptom of inflammation/tissue pain/muscle aches/headaches . . . . Can recognize this increase of voltage through experiments by placing a pair of electrodes [metallic Zn-metallic Cu] connected to a voltage measuring device (with measuring range from 0 volts to 2 volts) in the test tube (test tube has diameter greater than 20 mm), and this test tube contains the solution [0.9 % NaCl+0.2 ppm CuCl2+0.4 ppm ZnCl2+0.6 FeCl3+enough 100% water], gradually adding into this test tube a solution of 0.1% arginine phosphate (neutral)/0.1% arginine glutamate/0.01% EDTA Mg/0.01% triethylenetetramine glutamate/0.01% triethylenetetramine diethylenetriaminepentaacetate] until the obtained voltage drops sharply, the results are the amount of arginine phosphate/arginine glutamate used to sharply reduce the voltage being dozens of times greater than the amount of [EDTA Mg/triethylenetetramine glutamate/ triethylenetetramine diethylenetriaminepentaacetate] used to sharply reduce the voltage. The biological significance of these discoveries is that when the diseased bodies (body suffering from diseases caused by pathogenic microorganisms)/diseased tissues (infected tissues/inflammatory tissues) are depleted of the above chelators, the diseased bodies/diseased tissues/pain tissues/inflammation tissues, then after the above chelators are present in the diseased bodies/diseased tissues, thanks to the introduction of the present invention pharmaceuticals containing the above chelators into the diseased bodies/diseased tissues, will reduce pain/inflammation in the diseased bodies/diseased tissues. The mechanism of the reducing of inflammation/pain be above chelators is that they hold metal ions such as Cu ions/Zn ions/Fe ions/other metal ions (i.e. inactivate these metal ions) which are agents involved in the generation of high voltage currents (in conditions of depletion of the above chelators) of the electrode pairs in metalloproteins of the tissues/nerve tissues/brain tissue, thereby helping to reduce pain/inflammation.

Experiments measuring the voltage obtained from a pair of electrodes [metallic Zn-metallic Cu] (with the surface of each electrode having a fabric layer holding gel solution+polarizer (such as NaCl)) applied to the skin surrounding [inflamed tissue/infected tissue] which were injected with the solution containing above chelators/peroxide/high concentration glycerol, the voltage obtained from these tissues decreased sharply after injection of the above solutions. The biological significance of the above discoveries related to voltage is that when the diseased bodies (bodies suffering from diseases caused by pathogenic microorganisms)/diseased tissue (infected tissue/inflammatory tissue)) are depleted of above chelators, generates pain/inflammation in the diseased bodies/diseased tissue, then after above solution of [peroxide/high concentration glycerol/[high concentration glycerol+peroxide] present in the diseased bodies/diseased tissues thanks to the introduction of the present invention pharmaceuticals containing [peroxide/glycerol with high concentration glycerol/[glycerol with high concentration glycerol+peroxide] into the diseased bodies/diseased tissues, will reduce pain/inflammation in the diseased bodies/diseased tissues. The mechanism of reducing the inflammation/pain of high concentrations of glycerol in diseased tissues is that glycerol reduces the polarity of diseased tissues, causing high voltage currents (in conditions of depletion of the above chelators) in the electrode pairs in the metalloproteins in diseased tissues, thereby helping to reduce pain/inflammation. The mechanism of reducing of the inflammation/pain of peroxides in diseased tissues (such as infected tissues/necrotic tissues/cancerous tissues/tissues containing much polar fluid . . . )is peroxides oxidize metal ion complex crosslinks (such as crosslinks of Zn—Fe complex/Cu—Fe complex/Zn-Cu—Fe complex . . . ) in the structure of metalloproteins of pathogens (such as pathogenic microorganisms/cancer cells/toxins), cause these crosslinks to break and to convert into insoluble metal oxides, thereby reduce the voltage of the electrode pairs in the diseased tissues, helping to reduce pain/inflammation. Another important biological significances of the present invention pharmaceuticals containing [high concentration glycerol+peroxide]/[high concentration glycerol+peroxide+chelator] is the ability to treat diseased tissues (such as bacterial infection tissues/fungal infection tissues/necrotic tissues/dark-colored benign tumors/cancerous tumors (cancerous tissue)/diseased tissues containg high polarization-fluid tissues . . . ) with very high treatment effectiveness and very short treatment time (less than 24 hours to several days (several days for large-volume cancer tissues), by injecting small doses sequentially into different locations in large-volume cancer tissue), thanks to the pharmaceutical causing the diseased tissue to have the foam structure containing substances that promote the absorption of active ingredients of the drug, thanks to that, pathogens [pathogenic microorganisms/cancer cells that are rich in H2O2 decomposition catalysts]/toxins that are also rich in H2O2 decomposition catalysts] are quickly killed/destroyed, while healthy cells remain safe due to healthy cells are poor in H2O2 decomposition catalysts, this meaning is of prime importance in the science of treating diseased tissues, especially in the science of treating cancerous tissues because the pharmaceutical contains high concentration glycerol+peroxide]/[high concentration glycerol+peroxide+artificial substance. chelate] helps kill all cancer cells in cancer tissue in a very short time (less than 24 hours to a few days, a few days for large cancer tissues), this significance has prime important nature in the science of treating diseased tissues, especially in the science of treating cancerous tissues because the pharmaceutical contains high concentration glycerol+peroxide]/[high concentration glycerol+peroxide+chelator] help kill all cancer cells in cancer tissues in a very short time (less than 24 hours to a few days, a few days for large cancer tissues).

From the above discoveries, the invention has found a series of new elementary biological foundations in the biological world, and based on these elementary biological foundations, the invention has developed the pharmaceutical type with active ingredient being chelators with [Zn ions/other transition metal ions] holding feature (referred to as chelator drugs) to treat the symptoms as mentioned above/diseases as mentioned above/syndromes as mentioned above, The present invention chelator drugs operate by mechanisms completely different from those of contemporary pharmaceuticals for the treatment of above symptoms/above diseases/above syndromes. The present invention chelator pharmaceuticals aim to precisely attack pathogenic agents/pathogenic entities, therefore, the present invention chelator pharmaceuticals have outstanding effectiveness in disease prevention/disease treatment, and thanks to that, the pharmaceuticals have the ability to save lives in severe cases/critical cases of most diseases caused by pathogenic microorganisms, including dangerous diseases with high/very high mortality rates, especially the present invention chelator pharmaceuticals are very effective in treating cases of difficulty breathing/respiratory failure/acute respiratory failure in diseases caused by microorganisms. The chelators of the present invention chelator pharmaceuticals have the following effects and the mechanisms of action as following:
symptoms/neurological symptoms in diseases caused by pathogenic microorganisms and in some other diseases;
the thickening of blood/the thickening of tissue fluid/the thickening of alveolar fluid/blood clots in diseases caused by microorganisms and in and treatment of anaphylactic shock caused by drug injection/vaccination/infusion, and the treatment of cases anaphylactic shock in cases of anaphylactic shock caused by drug injection/vaccine injection/infusion;
shortness of breath/respiratory failure/acute respiratory failure in diseases caused by microorganisms and shortness of breath/respiratory failure/acute respiratory failure after disease;
cytokine storm;
most diseases caused by microorganisms including dangerous diseases with high (including Covid 19)/very high mortality rates;
severe cases/critical cases of diseases caused by pathogenic microorganisms (including severe cases/critical cases of dangerous diseases with high mortality rates (including Covid 19 disease)/very high);
infectious diseases that can easily spread;
weakened immune systems caused by chronic diseases/depletion of polyamine derivatives in the elderly;
prevent metastasis in patients with stage 3 and 4 cancer;
diseased tissues are bacterial infection tissues/necrotic tissues/fungal infection tissuea/tissues containing fluid with many toxins (tissue with proliferating cells)/benign tumors/cancerous tumors (tissue with cancerous cells);
diseases caused by external toxins/external neurotoxins;
some other different disease types;
infectious diseases are easily spread by using the active ingredients of chelators (mainly Ca aminopolycarboxylates) to create environmental treatment products that help prevent the spread of infectious diseases;
diseases caused by pathogenic microorganisms which they present in products outside the host body, by the use of active ingredients chelators (mainly polyamine aminopolycarboxylates) to create products for handling/preserving these products;
immune systems are easily weakened when the host is infected with diseases caused by microorganisms by using the active ingredient of the chelators to introduce into products which are beverages/processed foods/spices/prepared food/vitamin supplements/various medications.

From the discoveries related to chelators and the discoveries about physics effects in biology/about the new chemical bonds in biology, the invention has developed the pharmaceutical type which contains chelators and agents that creates strong physics effects in diseased tissues to treat the diseased tissues (referred to as chelator-strong physics effect pharmaceutical type) with the active ingredients are [chelators with [Zn ions/other transition metal ions]-holding feature+agents that produce strong physics effects in diseased tissue (such as glycerol at high concentrations)+low water content] to treat diseased tissues (such as bacterial infection tissues/necrotic tissues/fungal infection tissues/diseased tissue containing fluid with many toxins/benign tumors/cancerous tumors). This pharmaceutical type works by the mechanism of losing the ability to protect pathogenic agents/pathogenic entities in diseased tissues, which are tight structures/structures containing highly viscous fluid which these structures make the speed of dispersing the treatment ingredients in the treated tissues very slow, making the treatment of these diseased tissues difficult (especially for large volume diseased tissues). This pharmaceutical type produces a series of physics effects in the treated tissue that provide a series of benefits for the treatment of diseased tissues, these benefits are:
greatly increases the drug dispersion speed in diseased tissue;
temporarily replaces large amounts of water in diseased tissue with glycerol, which strongly inhibits the activity of pathogenic microorganisms in tissue infected with pathogenic microorganisms/cancer cells in cancerous tissue, but does not cause harmful to host tissues/host cells;
softens tissue, promotes tissue healing and helps reduce pain caused by dehydration in open wounds during tissue healing;
makes blood (containing many white blood cells/antibodies) circulate again in diseased tissue;
reduces the voltage of electrode pairs in tissues/nervous tissues in diseased tissue, reducing inflammation/pain;
weaken pathogenic microorganisms/cancer cells;
completely stops the activity of pathogenic microorganisms/cancer cells;
neutralizes the toxicity of toxins and neurotoxins in treated tissue;
inactivates sources of Zn/other heavy metals that pathogenic microorganisms/cancer cells need to multiply rapidly;
and many other benefits . . .

Because the above chelator-strong physics effect pharmaceutical type creates a series of strong physics effects in the treated tissue as mentioned above and brings a series of benefits as mentioned above, therefore, this pharmaceutical type is very effective in treating diseased tissues and help get a short time for treatment, while contemporary pharmaceuticals do not contain chelators with strong [Zn ions/other transition metal ions]-holding feature and do not contain agents that produce strong physics effects in the treated tissue, so contemporary pharmaceuticals that treat diseased tissues have many difficulties in treating diseased tissues (especially for large-volume diseased tissues).

From the discoveries related to chelators and the discoveries of physics effects in biology/related to new chemical bonds as mentioned above, along with the discoveries of other strong physics effects/the physics effects create the foam structure in the treated tissue, brings a series of other great benefits in the treatment of diseased tissues, from these discoveries, the invention has developed the pharmaceutical type that not only creates a series of effects and strong beneficial physics effects in the treated tissue as mentioned above, and also gives the treated tissue the foam structure, help causes pathogenic agents/pathogenic entities to locate in the thin walls created between tiny air bubbles and help create many other beneficial physics effects (such as the change in pressure of air bubbles continuously, helps increase the penetration of active ingredients into pathogenic agents/pathogenic entities that located in thin walls). The pharmaceuticals contains ingredients including [chelators with strong [Zn ions/other transition metal ions]-holding feature+agents that promote the speed of penetration into the treated tissue (such as high concentrations of glycerol)+agents that create the foam structure for the treated tissue being peroxide+low concentration water] (referred to as chelator-foam-strong physics effect pharmaceutical to treat diseased tissues (such as bacterial infection tissues/necrotic tissues/fungal infection tissue/disease tissues containing fluid with many toxins/benign tumors/cancerous tumors), chelator-foam-strong physics effect pharmaceuticalshave the same effects as chelator-strong physics effect drug mentioned above, at the same time, they have the additional effects as following:
kills microorganisms/cancer cells extremely rapidly by oxidizing Zn—Fe crosslinks/Zn—Cu—Fe crosslinks/other Zn-heavy metal crosslinks to convert them to metal oxides, causing these crosslinks to break, causing microorganisms/cancer cells to quickly die (while the host healthy cells are still safe (because the amount of H2O2 catalyst in the host healthy cells is much lower than the amount of H2O2 catalyst in the body of pathogenic microorganisms/cancer cells);
quickly oxidizes toxins in diseased tissue, quickly eliminating the toxicity of these toxins; creates the foam structure for diseased tissue so that pathogenic agents/pathogenic entities are located in the thin walls formed by tiny air bubbles, increasing the dispersion speed of active treatment ingredients in diseased tissue with the very high, and the contact between pathogenic agents/pathogenic entities in the treated tissue and the active treatment ingredients become very thorough, helping to very quickly kill the pathogenic microorganisms/cancer cells and helps quickly oxidize toxins in diseased tissue, causing these toxins to quickly lose their toxicity, helping to create a series of beneficial mechanical effects for treatment (such as the increase and decrease in the volume of air bubbles, and the air bubbles push each far away, increasing the drug permeability in the walls of the air bubbles/increasing the contact between pharmaceuticals and pathogens and toxins in tissue diseases . . . ), helping to effectively treat diseased tissues with a much shorter treatment time, and helping to reduce disease recurrence;
causing pathogenic agents/pathogenic entities to locate in thin walls created by tiny air bubbles (for pharmaceuticals chelator-foam-strong physics effect drugs), thereby accelerating the dispersion of the drug active ingredients to increase greatly in the diseased tissue, and causes the drug active ingredients to completely contact with all pathogenic agents/pathogenic entities in the treated tissue;
causes the drug active ingredients to penetrate strongly into the pathogenic agents/pathogenic entities in the thin walls due to the continuous change in pressure of the air bubbles that create the thin walls, thanks to that, the amount of drug active ingredients entering the pathogenic agents/pathogenic entities in the thin walls increases, and quickly kills the pathogenic entities in these thin walls.

### DETAILED DESCRIPTION OF THE INVENTION

The present invention pharmaceuticals have the active ingredients that are chelators with strong [Zn ions/other transition metal ions]-holding feature+(referred to as chelatordrugs), in which, which chelators with strong [Zn ions/other transition metal ions]-holding feature can hold more [Zn ions/other transition metal ions], those chelators have the more effective in [relieving symptoms of diseases caused by microorganisms/relieving post-disease syndromes/treating diseases caused by pathogenic microorganisms/treating diseases caused toxins and neurotoxins/treating cancers/treating weakened immune systems in chronic diseases (such as HIV/tuberculosis/chronic lung infections/chronic diseases of other diseases caused by pathogenic microorganisms/treating cancer stage 3.4/treating of weakened immune system in the elderly/treating the syndromes in the elderly/treating the secondary diseases caused by secondary pathogenic microorganisms . . . ], chelators with strong [Zn ions/other transition metal ions]-holding feature present invention chelator pharmaceuticals (please see abbreviations below are abbreviations for the substances mentioned above), typical as following:

polyamine aminopolycarboxylate is made up of polyamine which polyamine is Triethylenetetramine (TETA)/Tetraethylenepentamine (TEPA)/Pentaethylenehexamine (PEHA)/Branched polyethylenimine (branched PEI) with aminopolycarboxylic acid which aminopolycarboxylic acid is Ethylenediaminetetraacetic acid (EDTA)/Diethylenetriaminepentaacetic acid (DTPA)/triethylenetetramine-N, N, N′, N″, N′″, N′″-hexaacetic acid (TTHA)/Dibenzothiophene-1,3,6,8-Tetracarboxylic acid (DTCA)/ethylene glycol bis(2-aminoethyl ether)-N, N, N′, N′-tetraacetic acid (EGTA)/1,2-bis(2-aminophenoxy)ethane-N, N, N′, N′-tetra-acetic acid (BATPA)/1,4,7-triazacyclononane-1,4,7-triacetic acid (NOTA)/1,4,7,10-tetraazacyclododecane-1,4,7,10-tetraacetic acid (DOTA)/N, N′-bis(2-hydroxyethyl)ethylenediamine-N, N′-diacetic acid (HEEDA)/1,4,7,10,13-pentaazocyclopentadecane pentaacetic acid (PEPA)/1,4,7,10,13,16-hexaazocyclooctadecane hexaacetic acid (HEHA)/1,4,8,11-tetraazacyclotetradecane-1,4,8,11-tetraace-tic acid (TETA)/ triethylenetetraminepentaacetic acid (TETPA)/4,5-diphenyl-1,3-oxazol-2-yl)thio](phenyl)acetic acid (DOTPA)/1,4,7,10-tetraaxacyclododecane-1,4,7,10-tetramethylenephosphonic acid (DOTMP)/Ethylenediamine-N, N′-disuccinic acid (EDDS)/2-hydroxyethyliminodiacetic acid (HEIDA)/Glutamic acid diacetate (GLDA)/Methylglycinediacetic acid (MGDA)/1-aspartic acid N, N-diacetic acid (ASDA)/1,2-cyclohexylenediamine tetra-acetic acid (CDTA)/ethylenediamine-N, N′-bis(2-hydroxyphenylacetic acid) (EDDHA)/ethylene glycol bis(2-aminoethyl ether)-N, N, N′, N′-tetraacetic acid (EGTA)/[2-{4,7-biscarboxymethyl(1,4,7) triazacyclonona-1-yl-ethyl} carbonylmethylamino] acetic acid (NETA)/1,2-cyclohexylenedinitrilotetraacetic acid (CyDTA)/triethylenetetraaminehexaacetic acid (TTHA)/1,4,7,10-Tetraazacyclododecane-1,4,7,10-tetraacetic acid (DO3A) /6-amino-6-methylperhydro-1,4-diazepinetetraacetic acid (AAZTA);

Mg aminopolycarboxylate/Ca aminopolycarboxylate is made up of [Mg derivative/Ca derivative] with aminopolycarboxylic acid as [EDTA/DTPA/TTHA/DTCA/EGTA/BAPTA/NOTA/DOTA//HEEDA//PEPA/HEHA/TETA /TETPA/DOTPA/DOTMP/EDDS/HEIDA/GLDA/MGDA/ASDA/CDTA/EDDS/EDDHA/DOT A/EGTA/NETA/CyDTA/GEDTA/TTHA/DO3A/AAZTA];

the salt made up of polyamine with acid that has the sulfur-containing functional group in the molecule, is the salt made up of the polyamine [TETA/TEPA/PEHA/PEI branches] with the acid that has the sulfur-containing functional group in the molecule that is [sulfonic acid/sulfuric acid /sulfurous acid/sulfenic acid/sulfinic acid/hydrogen sulfide acid/hyposulfurous acid];

the salt made from polyamine with acids having at least 2 carboxyl-containing functional groups in the molecule are salts made from polyamines [TETA/TEPA/PEHA/HEPA/HEEDA/ /PEPA/PEI/branched PEI] with acids having at least at least 2 carboxyl-containing functional groups in the molecule are [gluconic acid/fumaric acid/itaconic acid/glutamic acid/aspartic acid/carbonic acid/phosphoric acid/citric acid/succinic acid/malic acid];

the salt made from polyanine with acids having at least 2 hydroxyl-containing groups in the molecule are salts made up from polyamines are TETA/TEPA/PEHA/branche PEI] with acids having at least 2 hydroxyl-containing functional groups in the molecule are ascorbic acid/gallic acid/tannic acid;

One unit of the above synthetic chelator of the chelator pharmaceuticals is capable of holding 80 units to more than 200 units of [Zn ions/other transition metal ions], while on unit natural chelator (such as one unit of lysine phosphate/lysine chloride/arginine phosphate/arginine chloride) is capable of holding one unit (or few units) of [Zn ions/other transition metal ions], these help to understand that the chelators of the present invention chelator pharmaceuticals have the ability to quickly relieve symptoms/to prevent the appearance of post-disease syndromes/to weaken pathogenic microorganisms/to strengthen the immune system/to prevent the cancer metastasis/to inactivating toxins and neurotoxins/to recover the host body from many different diseases . . . far excess of these abilities of natural chelators which present in the host body.

Chelators of chelator pharmaceuticals/chelator-strong physical effect pharmaceuticals/chelator—foam-strong physical effect pharmaceuticals of the present invention when present in the host body/diseased tissues of the host (host is animal/human) have the following effects and have mechanisms of action as following:
make to rapidly lose the symptoms in diseases caused by pathogenic microorganisms (including dangerous symptoms/dangerous neurological symptoms), thanks to chelators of drug to separate [Zn/other transition metals]-rich products (released from pathogens and their fragments, referred to as from pathogenic microorganisms) which are [toxins/neurotoxins] and are the agents that cause symptoms from the host metalloproteins, and then chelators hold [Zn/other transition metal-rich products] which are removed from patient's body through urine;
make to rapidly lose the toxicity of toxins/neurotoxins (including toxins/neurotoxins produced by pathogenic microorganisms in the patient's body/external toxins or external neurotoxins from the outside entering the host's body), thanks to chelators of drug to separate toxins/neurotoxins from the host's metalloproteins, and then chelators which held toxins/neurotoxins that are removed from the patient's body through urine;
inactivates pathogenic microorganisms, thanks to chelators of drug to encapsule Zn ions/transition metal ions in metalloproteins on the surface of pathogenic microorganisms (on the surface of spike protein, for pathogenic viruses) , causing pathogenic microorganisms to lose the ability to attach to metalloproteins on the host cell membrane/on receptors of the host cell membrane, causing pathogenic microorganisms to lose the ability to attack the host cells/to lose the ability to penetrate into the host cells;
make Zn derivatives/other metal derivatives in the host body which are the sources of raw materials required by pathogenic microorganisms to convert into the form of Zn chelate /transition metal chelate, which microorganisms cannot use for their rapid growth/their rapid multiplication, causes to weaken pathogenic microorganisms, while the host's healthy cells can still be used because the host's healthy cells with the multiplying very slowly/growing very slowly;
make to rapidly lose the cytokine storm, thanks to chelators of drug to separate [Zn/other transition metal]-rich products (from pathogenic microorganisms) from metalloproteins on the host cell membrane/on receptor on the host cell membrane, and then chelators which held toxins/neurotoxins that are removed from the patient's body through urine, makes white blood cells no longer mistake these host cells as pathogenic microorganisms (the surface of pathogenic microorganisms has the high density of Zn ions/transition metal ions which helps them to adhere to metal ions in host cell metalloproteins), so white blood cells no longer attack these host cells, thereby the cytokine storm no longer exists;
make to rapidly lose the symptoms of blood thick/tissue fluid thick/alveolar fluid thick, thanks to chelators of drug to quickly dissolve the suspensions composed of Zn-blood complexes that are formed from Zn-rich products (from microorganisms) and their fragments with the blood;
make to rapidly lose the blood clots, thanks to chelators of drug to breaks the Zn crosslinks on the surface of platelets (which are made up of Zn-rich products (from microorganisms and their fragments) which causes the platelets to stick to each other, and causes the above Zn-blood complex suspension particles to stick to the surface of platelets, thereby dissolving blood clots;
make to rapidly lose the neurological symptoms (such as symptoms of loss of taste/loss of appetite/sore throat/cough/muscle pain/headache/fever . . . ), thanks to chelators of drug to strongly reduce the voltage of electrode pairs in metalloproteins of nerve tissues, helps bring the voltage of the electrode pairs of nerve tissues to the normal state, thereby eliminating neurological symptoms/inflammatory symptoms;
make to rapidly lose the symptoms of respiratory failure/acute respiratory failure, thanks to chelators of drug to hold Cu ions of free Cu derivatives in the patient's blood (free Cu derivatives which appear from the diseased body that is depleted of polyamine derivatives which have the ability of holding the Cu ions, and the Cu ions of these free Cu derivatives adhere to the Fe ions in metalloproteins of [red blood cells/alveolar cells/other host cells], cause these cells to lose the ability to exchange O2, causing the patient to suffer respiratory failure/acute respiratory failure), causes [red blood cells/alveolar cells/other host cells] to quickly return O2 exchange normally, thereby helping the patient quickly restore respiratory activity;
inactivates the metastatic cancer cells that have left the cancer tissue, thanks to chelators of the drug to encapsule Zn ions/transition metal ions in metalloproteins on the surface of the metastatic cancer cells that have left the cancer tissue, causing the metastatic cancer cells that have left the cancer tissue to lose the ability to attach to metalloproteins on the host cell membrane/on receptors on the host cell membrane, make the metastatic cancer cells that have left the cancer tissue to lose their ability to metastasize to healthy cells/healthy tissues;
reduces/clears the accumulation of [Zn ion/Zn ion-other transition metal ion complexes] in nervous tissues/brain tissues, thereby reducing/eliminating a number of syndromes (such as post-Covid/dementia syndrome or tremor syndrome or cataract syndrome in the elderly (syndromes in the elderly appear due to the amount of polyamine derivatives in the elderly body gradually decreasing over time), causing the accumulation of [Zn ion/Zn ion-other transition metal ion complexes] in the elderly less to be washed by polyamine derivatives;
strongly reduces the voltage of electrode pairs in metalloproteins of living entities (including healthy cells/healthy tissues/pathogenic microorganisms/syncytia pathogenic microorganisms/cancer cells/cancerous syncytia) and strongly reduces the polarity of metalloproteins of living entities in the patient's body, thereby paving the way for various pharmaceuticals when used at the same time with chelator to increase the amount of penetration into the living entities, thanks to which the doses of different pharmaceuticals are sharply reduced and at the same time the effectiveness of these different pharmaceuticals is greatly increased even though the doses of these different pharmaceuticals that are very small; especially drastically reducing the dose of antibiotics, and at the same time being able to reuse antibiotics that are resisted by pathogenic microorganisms;
solution containing [chelators with strong [Zn ions/other transition metal ions]-holding feature+high concentration polyol]/solution containing [chelators with strong [Zn ions/other transition metal ions]-holding feature+peroxide+high concentrations of polyol] when present in cancerous tissue to break transition metal crosslinks/Zn-other transition metal complex crosslinks in metalloproteins/between metalloproteins of [cancer cells/cancer syncytium/cancer tissue] (which are crosslinks that cause the size of [cancer cells/cancer syncytium/cancer tissue] to increase continuously), causing cancer cells to die/cancer syncytium to disintegrate/cancer tissue to decrease in size, so up to now there are no pharmaceuticals in the form of the solution containing [chelators with strong [Zn ions/other transition metal ions]-holding feature+high concentration polyol]/solution containing [chelators with strong [Zn ions/other transition metal ions]-holding feature+peroxide+high concentrations of polyol] to treat the cancerous tissues (also to treat the benign tumors);
strengthens the host's immune system, thanks to the chelators of drug to integrate into [Cu ion/ Mg ion/Ca ion] in the structure of [antibacterial alkaline peptide/antibody protein/microbial killing protein in in white blood cells], causing [antibacterial alkaline peptide/antibody protein/microbial killing protein in white blood cells] to greatly increase the ability to kill pathogenic microorganisms, thereby participating in strengthening the immune system (the host strong immune system is also due to the amount of polyamine derivatives which still have the active of holding [Zn ions/other transition metal ions] present in the host body); especially strengthens the weakened immune systems of people with [HIV/tuberculosis/some other chronic diseases/cancer in stage 3-4] (pathogenic microorganisms/pathogenic microorganisms syncytia pathogenic/cancerous cells/cancerous syncytial/cancerous tissues which are biological factories that take up [Zn ions/other transition metal ions/citrate/succinate/malate/gallate] in the host tissues, then releases into the host body the [Zn ions/other transition metal ions/citrate/gallate] as [Zn/other transition metals/citrate/succinate/malate/gallate]-rich products as waste products. deficiency/depletion of chelating agents with [Zn ion/transition metal ion] retention properties, weakening the host immune system); makes to diminish/to deplete chelators with [Zn ions/other transition metal ions]-holding feature, makes to weaken the host immune system);
washes (i.e. removes) the accumulation of [Zn ion/Zn ion-other heavy metal ion complexes/other heavy metal ions] in the host's tissues/nerve tissue/brain tissue, helping to prevent the generation/to lose (to lose for the present invention chelator pharmaceuticals) post-illness syndromes (such as post-Covid 19 syndrome/brain fog syndrome in people who have had Covid 19)/to reduce [dementia syndrome/tremor syndrome/cataracts . . . ] in the elderly, and also thanks to this “washing mechanism”, the host's body has better health after illness/when the host's body is elderly body.
produces a series of other physics effects along with the effect of reducing the voltage of electrode pairs in metalloproteins of living entities that cause disease and of living entities of the host, harming pathogenic entities, but makes the host's entities stronger, thereby making the host's body healthier/host's immune system stronger/host in the more comfortable state/less painful/less sick.

Because all pathogenic microorganisms use high concentrations of [Zn ions/other transition metal ions] on their surfaces (on spike proteins for pathogenic viruses) to attach to metalloproteins on the host cell membrane/receptors on the host cell membrane, metalloproteins of pathogenic microorganisms accumulate a large amounts of Zn ions/other transition metal ions (due to pathogenic metalloproteins are rich in cysteine/polar amino acids that create segments to attract and to accumulate Zn ions/other transition metal ions), after accumulating many Zn ions/other transition metal ions, pathogenic microorganisms and their fragments release [Zn derivatives/other transition metal derivatives/Zn-other transition metals complex derivatives/peptides and proteins rich in [Zn/other transition metals] (referred to as [Zn/other transition metals]-rich products from pathogenic microorganisms) into the host body, these [Zn/other transition metals]-rich products (which are also toxins/neurotoxins) rapidly reduce the amount of chelators with [Zn ions/other transition metal ions]-holding feature in the first phase, rapidly reduces the amount of chelating agent with chelators with [Zn ions/other transition metal ions]-holding feature which still have the activity of holding of [Zn ions/other transition metal ions]; in the second phase, these chelators are depleted due to hold Zn ions/other transition metal ions, the Zn/other transition metals]-rich products bind to the metalloproteins of cells/tissues/nerve tissues/brain tissue of the host that generates the symptoms; the third phase is the recovery phase, during this phase the host body provides new chelators (mainly polyamine derivatives such as histidine/lysine/arginine/ornithine/agmatine/putrescine/cadaverine/ derivatives). spermidine/spermine . . . Mg-porphyrin) and these new chelators help gradually separate [Zn/other transition metal]-rich products from pathogenic microorganisms from host metalloproteins, gradually reducing the intensity of symptoms, helps the host body recover, however, the recovery by these new natural chelators takes a long time (weeks to months for unvaccinated patients) and as [Zn/other transition metal]-rich products from pathogenic microorganisms are not completely separated from the host metalloproteins by these new chelators, causing post-disease syndrome.

The present invention chelator pharmaceuticals contain chelators with very strong [Zn ion/other transition metal ion] holding feature, which can be detected through one chelators unit of the present invention chelator pharmaceutical can dissolve 80 units to 200 units of the suspension composed of Zn-blood complex, while natural chelators are present in the host, must be required about 100 units of natural chelators to dissolve 100 units of suspension composed of Zn-blood complex (as mentioned in the background of invention), this ability of the chelators of the present invention chelator pharmaceuticals helps to understand why the present invention chelator pharmaceuticals cause rapid disappearance of symptoms in serious cases of diseases caused by pathogenic microorganisms surprisingly (such as Covid 19 (on human)/influenza (on human)/viral fever (on human)/avian influenza (clinical trial on chicken), with symptoms significantly reducing approximately 3-4 hours after taking the first oral dose/approximately 20 minutes after the first injection dose, and the patient achieves the pleasant felling clearly, while before using the present invention chelator pharmaceuticals the patient (human) had [severe sore throat/severe cough/severe muscle pain/loss of taste/loss of appetite/fatigue/headache . . . in very high levels which are unbearable levels. For example, the pharmaceutical to treat Covid 19 with each dose for adults contains 1.4 g Mg EDTA/0.8 g triethylenetetramine diethylenetriaminepentaacetate, the pharmaceutical is taken 3 times a day for 7 days, and is taken ½ above doses for next 8 days to prevent the occurrence of post-Covid syndrome, resulting in patients with severe Covid-19 (including patients who have not received any Covid-19 vaccine shot/patients who are older than 75 years old), 4 hours later after taking the first dose, the symptoms were significantly reduced, after 48 hours the remaining symptoms were negligible, the next few days the disease was cured, and after treatment, the post-Covid syndrome did not appear. In cases of Covid 19 with lung infection, patients use Bactrim at a dose equal to ¼ of the normal dose of Bactrim (because when using antibiotics at the same time as chelator pharmaceutical, makes the effectiveness of antibiotics to increases very strongly, and makes the dose of antibiotics which are needed to use much lower than that the normal doses of antibiotics).

Clinical trials on animals/humans for the treatment of certain diseases caused by microorganisms with the present invention chelator pharmaceuticals containing polyamine aminocarboxylate/salt is made up of polyamine with acids having the sulfur-containing functional group in the molecule/Mg aminopolycarboxylate/Ca aminopolycarboxylate (Ca aminopolycarboxylate for injection route), showing that the chelators of the present invention chelator pharmaceuticals are exceptionally effective in treating symptoms of diseases caused by microorganisms, and has outstanding treatment effectiveness in the treatment of diseases caused by microorganisms, chelators of the present invention chelator pharmaceuticals containing the above chelators have the following effects:
make to quickly lose the symptoms;
make to weaken the pathogenic microorganisms;
to incorporate into Cu ions in [antibacterial alkaline peptides/antibody proteins/pathogen-killing proteins in white blood cells], induce these peptides/proteins to increase their ability to kill pathogenic microorganisms;
wash away the accumulation of [Zn ions/transition metal ions/Zn-other transition metal ion complexes] in tissues/nervous tissue/brain tissue, helping to relieve post-morbid syndrome;
make the toxins or the neurotoxins to lose their toxicity;
make to generate a series of other benefits as mentioned in the background of invention.

The clinical treatment effectiveness of present ionvention chelator pharmaceuticals for the treatment of above diseases, along with the clinical treatment effectiveness on animals that are infected with toxins/neurotoxin as mentioned in above background of invention, help predict the present invention chelator pharmaceuticals especially drugs whose contain chelators being polyamine aminopolycarboxylate (such as polyamine aminopolycarboxylate made from [TETA/TEPA/PEHA/PEI branched with EDTA/DTPA/TTHA/DTCA /EGTA/BAPTA/NOTA/DOTA//HEEDA//PEPA/HEHA/TETA/TETPA/DOTPA/DOTMP/EDD S/HEIDA/GLDA/MGDA/ASDA/CDTA/EDDS/EDDHA/DOTA/EGTA/NETA/CyDTA/GEDT A/TTHA/DO3A/AAZTA] which are the pharmaceuticals that can promptly save the lives of severe cases/critical cases of dangerous diseases caused by microorganisms with high/very high mortality rates (such as Covid 19/avian influenza/Ebola disease/Marburg/smallpox/chickenpox/tetanus/rabies/Aids/Anthrax/ . . . blood infection/brain infection).

The active ingredient polyamine aminopolycarboxylate, such as polyamine aminopolycarboxylate which is made from [polyamine is TETA/TEPA/PEHA/PEI branched with aminopolycarboxylic acid is EDTA/DTPA/TTHA/DTCA/EGTA/BAPTA/NOTA/DOTA//HEEDA//PEPA/HEHA /TETA/TETPA/DOTPA/DOTMP/EDDS/HEIDA/GLDA/MGDA/ASDA/CDTA/EDDS/EDDHA /DOTA/EGTA/NETA/CyDTA/GEDTA/TTHA/DO3A/AAZTA] are chelators that have the effect of loss of toxicity of toxins/neurotoxins (including toxins/neurotoxins produced by pathogenic microorganisms in the patient's body and external toxins/external neurotoxins, such as from rat poison/pesticides/venoms of poisonous animals/poisonous plants/poisonous mushrooms/toxins produced by biotechnology/some organic toxins and other inorganic toxins), the active ingredient triethylentetramine diethylenetriaminepentaacetate and some other polyamine aminopolycarboxylates as mentioned above have been used for clinical treatment of small-size animals (such as Boraras Micros fish/flies and fly larvae/crickets/earthworms//sea worms/sipunculus nudus/chickens/rats) are contaminated with many types of toxins/neurotoxins that are the active ingredients of rat poisons/pesticides/poisonous plants . . . , and all achieved results that helped experimental animals not die, even they are infected with the doses of toxins/neurotoxins being many times to dozens of times of the minimum doses that cause they to die.

Recognizing the ability to lose the toxicity of toxins/neurotoxins by the active ingredient being triethylentetramine diethylenetriaminepentaacetate through clinical trials on Boraras Micros fish as follows:
keeping fish in water with [5 ppm Nereistoxin+50-300 ppm triethylenetetramine diethylenetriaminepentaacetate] fish lived for many days (up to weeks), while keeping fish in water with 5 ppm Nereistoxin fish died after 30 minutes;
keep fish in water with [1.25 ppm flocoumafen (¼ tablet of rat poison (with 0.005% flocoumafen) in 1 liter of water)+50-300 ppm triethylenetetramine diethylenetriaminepentaacetate], fish lived for days (up to weeks), while keeping fish in water containing 1.25 ppm flocoumafen, fish died after 36 hours;
keeping fish in water with [0.3 ppm Bromadiolone+50-300 ppm triethylenetetramine diethylenetriaminepentaacetate], fish lived for many days, while keeping fish in water with 0.3 ppm Bromadiolone, fish died within 12 hours;
keeping fish in water with [0.2 ppm Methylamine Avermectin 50-300 ppm triethylenetetramine diethylenetriaminepentaacetate], fish lived for many days, while keeping fish in water with 0.2 ppm Methylamine Avermectin, fish died in 12 hours;
keeping fish in water with [2.5 ppm Emamectin benzoate 50-300 ppm triethylenetetramine diethylenetriaminepentaacetate], fish lived for days, while keeping fish in water with 2.5 ppm Emamectin benzoate, fish died within hours;
keeping fish in water with [1.0 ppm phenylperazole+50-300 ppm triethylenetetramine diethylenetriaminepentaacetate], fish lived for many days, while keeping fish in water with 1.0 ppm phenylperazole, fish died within a few hours;
keeping fish in water with [[0.2 ppm Alpha Cypermethrin+50-300 ppm triethylenetetramine diethylenetriaminepentaacetate], fish lived for many days, while keeping fish in water with 0.2 ppm Alpha Cypermethrin, fish died within a few hours;
keeping fish in water with [0.3 ppm CuCl2+20 ppm ZnCl2+20 ppm FeCl3], the fish lived for many days, while keeping fish in water with [0.3 ppm CuCl2+20 ppm ZnCl2], the fish died in a few hours; these experiments help identify that during the hemorrhagic stage of dengue fever, the patient's fever reduces and feels comfortable due to the increased concentration of Fe derivatives in the blood (from broken red blood cells);
keeping fish in water with 2000 ppm triethylenetetramine diethylnenepentaacetate/3000 ppm Mg EDTA/2000 ppm Cu EDTA/12000 ppm Ca EDTA/[10000 ppm+2500 ppm CuCl2], fish lived for many days, while keeping fish in water with 1000 ppm 2 Na EDTA/0.5 ppm CuCl2/[0.3 ppm CuCl2+20 ppm ZnCl2], fish died in a few hours, these experiments help identify the toxicity threshold of chelators to host cells (host is animal/human), and Ca EDTA/Ca DTPA are highly safe and help to treat the diseases caused by infection of heavy metal derivatives//radioactive metal derivatives with extreme high infection levels.

Recognizing the ability to Inhibit/to kill pathogenic microorganisms of the active ingredient triethylenetetramine diethylenetriaminepentaacetate by clinical experiments on Boraras Micros fish as follows: use a number of Boraras Miros fishes that died after 3 days (about 100 g), then put this amount of dead fishes into 10 liters of water, and keep the living fishes for 2 days in this 10 liters of water, so that all fishes in this 10 liters of water are infected with pathogenic microorganisms (from 100 g of dead fish after 3 days), then add 1 liter of water containing the above dead fishes to each large beaker, with each beaker contains about 30 living fish; then use 1 beaker as a control experiment, the remaining beaker add substances so that each cup contains 10 ppm Ampicillin/20 ppm Bactrim/20 ppm Ampicillin/40 ppm Bactrim/100 ppm triethylenetetramine diethylenetriaminepentaacetate/300 Mg EDTA/600 ppm Ca EDTA/[50 ppm Cu ETDA+100 triethylenetetramine diethylenetriaminepentaacetate]/[0.3 ppm Ampicillin+50 ppm triethylenetetramine diethylenetriaminepentaacetate]/[0.6 ppm Bactrim+50 ppm triethylenetetramine diethylenetriaminepentaacetate]; the results are after 6 days all fishes in the control experimental beaker/beaker containing [10 ppm Ampicillin/20 ppm Bactrim/20 ppm Ampicillin/40 ppm Bactrim] died and the water in these beaker became cloudy; and 6 days all fishes in beaker containing [100 ppm triethylenetetramine diethylenetriaminepentaacetate/300 Mg EDTA/600 ppm Ca EDTA/50 ppm Cu ETDA+100 triethylenetetramine diethylenetriaminepentaacetate/[0.3 ppm Ampicillin+50 ppm triethylenetetramine diethylenetriaminepentaacetate]/[0.6 ppm Bactrim+50 ppm triethylenetetramine diethylenetriaminepentaacetate] lived and the water in these beaker were clear.

The above results also indicate that aminopolycarboxylate polyamines when present in [blood/tissue fluid/alveolar fluid] of the host with the concentrations that are quite low (above 30 ppm for aminopolycarboxylate polyamines made from TEPA/PEHA/branched PEI) with DTPA, and above 60 ppm for polyamine aminopolycarboxylate formed from TETA with DTPA), the ability to inhibit/to kill pathogenic bacteria far exceeds this ability of Bactrim with the concentration of 10 ppm/Ampicillin with the concentration of 20 ppm in [blood/in tissue fluid/alveolar fluid] of the host.

In particular, drugs with the ingredients [polyamine aminopolycarboxylate+antibiotic active ingredient]/[polyamine aminopolycarboxylate+salt made from [polyamine with acid having the sulfur-containing functional group in molecule] are pharmaceuticals that kill bacteria very strongly, therefore, they are the outstandingly effective pharmaceuticals in the treatment of diseases caused by pathogenic microorganisms, and help greatly reduce the doeses of active ingredients of antibiotics/do not have to use antibiotics, and at the same time, these pharmaceuticals bring a series of other benefits as mentioned above (such as quickly reducing symptoms/cleansing the patient body from the accumulation of Zn ion or other transition metal ions/reducing pain and inflammation . . . )

Clinical trials on diseased tissues (e.g. bacterial infection tissues/fungal infection tissues/Fluid-filled tissue contains many toxins//benign tumors/cancerous tumors) by chelator-strong physics effect pharmaceuticals with the active ingredients being [chelators with strong [Zn ions/other transition metal ions]-holding feature+high concentrations of polyols]/[chelator-foam-strong physics effect pharmaceuticals] with the active ingredients being chelators with strong [Zn ions/other transition metal ions]-holding feature+high concentrations of polyols+peroxide], showing that these present invention pharmaceuticals have outstanding effectiveness in the treatment of the above diseased tissues, the above-mentioned pharmaceuticals have outstanding effectiveness in treating diseased tissues thanks to the strong physics effects that are generated in the treated tissue/[strong physics effects that are generated in the treated tissue+foam structure that are created in diseased tissue], greatly increases the speed of decomposition of the active ingredients of the pharmaceuticals, thereby overcoming the protection of tight structures/highly viscous fluid-containing structures of diseased tissues, which are the structures that protect pathogenic entities/pathogenic agents inside the diseased tissue. Such as the treatment for the severely infected tissues (on animal/human) by topical application of the chelator pharmaceutical solution “chelator-strong physics effect pharmaceutical” containing [70% glycerol+0.4% EDTA Cu+1 , 2 g EDTA Mg+3.6 g EDTA Ca+100% water] onto open wound on infected tissue (apply 4 times a day for the first days, and 2 times in the following days, and apply until the open wound heals (about 5-7 days), the infection is significantly reduced after 1 day, after 3 days the infection is gone, after 5 to 7 days the open wound heals with little scarring, the pharmaceutical generates the pain relief effect (only creates the mild burning sensation on open wounds for the first few minutes after application).

Chelator-foam-strong physics effect pharmaceutical type has the effects of Chelator-strong physics effect pharmaceutical type, in addition the chelator-foam-strong physics effect pharmaceutical type also has the following effects and benefits:
pushes high viscous fluid (containing many pathogenic microorganisms/toxins/nutrients for pathogenic microorganisms) out of infected tissue;
rapid dispersion of glycerol and of treatment ingredients into diseased tissues;
temporary replacement of large amounts of water in [host tissue/host cells/pathogenic microorganism body] with glycerol to inhibit the activity of pathogenic microorganisms but does not harm the host tissue/host cells;
softens tissue, promotes tissue healing and helps reduce pain caused by dehydration in open wounds during tissue healing;
causes blood (containing many white blood cells/antibodies) to re-circulate in diseased tissue;
increases the dispersed speed on of the active ingredients of pharmaceutical in the treated tissue;
creates thorough contact between the active ingredients of pharmaceutical and the pathogenic agents/pathogenic entities in the treated tissue, thereby quickly inactivating the pathogenic agents/quickly killing the pathogenic entities in treated tissue;
creates many other useful physics effects which help treated tissues to recover quickly;
does not have to cut severe diseased tissue from the patient's body;
just clean the open wound on the diseased tissue once before using pharmaceutical;
helps reduce the pain and reduce the inflammation (because chelators and polyols strongly reduce the voltage of electrode pairs in host metalloproteins in the treated tissue);
few side effects due to chelator/glycerol almost are not toxic to healthy host cells at the concentrations of chelator/glycerol for treatment of diseased tissues;
less recurrence, because the active ingredient of the pharmaceutical is evenly dispersed in the diseased tissue, and penetrates pathogenic agents/pathogenic entities with the large amounts;
easy to perform and low treatment cost;
less scarring and fewer sequelae after treatment, thanks to glycerol cleans the treated tissue/softens the treated tissue/makes the treated tissue to increase the elasticity/makes the blood to recirculate strongly in the treated tissue . . . , thanks to that, the treated tissues recover quickly;

The clinical treatment effectiveness of the above-mentioned diseased tissues is so outstanding which help to predict that the treatment of most diseased tissues will become easy thanks to the treatment of diseased tissues with the above present invention pharmaceuticals, in which thanks to the strong physics effects produced in the treated tissue by this chelator drugs-strong physical effect pharmaceutical type/this chelator drugs-foam- strong physical effect pharmaceutical type of present invention, which have outstanding effectiveness that far exceeds the effectiveness of contemporary pharmaceuticals (which are pharmaceuticals that do not produce physics effects in the treated tissue/produce physics effects that are not strong enough in the treated tissue; the results of successful clinical treatment of many different types of diseased tissues (in humans) with the above pharmaceuticals help from now on the treatment of most diseased tissues will be easily by the treatment of diseased tissues with these the present invention pharmaceuticals, these pharmaceuticals also bring a range of other benefits such as:
does not have to cut severe diseased tissue from the patient's body;
easy to carry out for treatment of diseased tissues;
only need to clean the open wound on the diseased tissue once before using the pharmaceutical;
few side effects (because the active ingredients of the pharmaceutical are not toxic/less toxic to healthy cells/healthy tissues during treatment and after treatment);
less recurrence (due to the active ingredient dispersing evenly in the diseased tissue);
low treatment costs;
less sequelae after treatment and less scarring after treatment;
help avoid the infection of secondary pathogenic microorganisms on treated tissues.

Chelator-strong physics effect pharmaceutical type has the effects of chelators of parmaceutical type in treated tissues, in addition the Chelator-strong physics effect pharmaceutical type also has the following effects and following benefits:
pushes the high viscous fluid (containing many pathogenic microorganisms/toxins/)nutrients for pathogenic microorganisms) out of infected tissue;
su′ phân tán nhanh chóng cua glycerol và các ch\hat{à}t di\hat{è}u tri vào các mô bênh;
rapid dispersion of glycerol and treatment ingredients into diseased tissues;
temporary replacement of large amounts of water in [host tissue/host cells/microbial body] with glycerol which help inhibit the activity of pathogenic microorganisms but does not harm the host cells/host tissues;
softens tissue, promotes tissue healing and helps reduce pain caused by dehydration in open wounds during the tissue healing;
causes blood (containing many white blood cells/antibodies) to recirculate in treated tissues;
increases the speed of dispersion of the active ingredients of the pharmaceutical in the treated tissues
creates thorough contact between the active ingredients of pharmaceutical and the pathogenic agents/pathogenic entities in the treated tissue, thereby quickly inactivating the pathogenic agents/quickly killing the pathogenic entities in treated tissue;
creates many other useful physic effects to help the treated tissues to recover quickly;
does not have to cut serious diseased tissues from the patient's body;
just clean the open wound on the diseased tissue once before using the pharmaceuticals;
helps reduce pain and inflammation (because chelating agents and polyols strongly reduce the voltage of electrode pairs in host metalloproteins in the treated tissue);
few side effects due to chelating agents/glycerol being virtually non-toxic to healthy host cells at chelating agent/glycerol concentrations in the treated tissue;
less recurrence, because the active ingredient of the pharmaceutical is evenly dispersed in the diseased tissue, and penetrates pathogenic agents/pathogenic entities with the large amounts;
easy to perform and low treatment cost;
less scarring and fewer sequelae after treatment, thanks to glycerol cleans the treated tissue/softens the treated tissue/makes the treated tissue to increase the elasticity/makes the blood to recirculate strongly in the treated tissue . . . , thanks to that, the treated tissues recover quickly;

From the discoveries related to chelators and the discoveries of physics effects in biology/new chemical bonds as mentioned above, along with the discoveries of other strong physics effects, thanks to the creation of the foam structure in the treated tissue which bring a series of other great benefits in the treatment of diseased tissues, also creates a series of powerful and beneficial physics effects in the treated tissue as mentioned above, also gives the treated tissues the foam structure, causes pathogenic agents/pathogenic entities to locate in the thin walls that are created between tiny air bubbles, along with many other beneficial physics effects are created (such as a change in pressure of air bubbles continuously that helps increase the penetration of active ingredients of pharmaceutical into pathogenic entity bodies/pathogenic agents which are located in thin walls). These pharmaceuticals contain ingredients including::—[chelator with [Zn ions/other transition metal ions]-holding feature+agents that promote the speed of the penetration of treatment ingredients in the treated tissue (such as high concentrations of glycerol)+low water content] (referred to as chelator-strong physical effect pharmaceutical);—[chelator with [Zn ions/other transition metal ions]-holding feature+agents that promote the speed of the penetration of treatment ingredients in the treated tissue (such as high concentrations of glycerol)+agents creates the foam structure for the treated tissue (such as peroxides_+low water content] (referred to as chelator-foam-strong physical effect pharmaceutical); these two pharmaceuticals are used for the treatment the diseased tissues (such as bacterial infection tissues/necrotic tissues/fungal infection tissues/diseased tissues containing fluid with many toxins/benign tumors/cancerous tumors), these two pharmaceutical types have the following effects as the chelator pharmaceutical type, at the same time, they have additional effects (as mentioned in Background of invention, which are re mentioned below in order to know the valuable benefits of pharmaceutical type containing [chelator with [Zn ions/other transition metal ions]-holding feature+agents that promote the speed of the penetration of treatment ingredients in the treated tissue (such as high concentrations of glycerol)+agents creates the foam structure for the treated tissue (such as peroxides_+low water content] in treating the diseased tissues with tight structures/structures containing high viscous fluid):
kills microorganisms/cancer cells extremely rapidly by oxidizing Zn—Fe crosslinks/Zn—Cu—Fe crosslinks/other Zn-heavy metal crosslinks to covert to the metal oxides, causing these crosslinks to break, causing microorganisms/cancer cells to quickly die (while healthy cells are still safe (because the amount of H2O2 catalyst in healthy cells is much lower than the amount of H2O2 catalyst in the body of pathogenic microorganisms/cancer cells);
quickly oxidizes the toxins in diseased tissue, make to quickly eliminate the toxicity of these toxins;—creates the foam structure for diseased tissue so that pathogenic agents/pathogenic entities which located in the walls formed between tiny air bubbles, increasing the dispersion speed of the active ingredients of pharmaceutical in diseased tissue to the very high levels, and the contact between the pathogenic agents/pathogenic entities in the treated tissue and the active ingredients of pharmaceutical become very thorough, helping to very quickly kill the pathogenic microorganisms/cancer cells and helping to quickly oxidizes the toxins in diseased tissue, causing these toxins to quickly lose their toxicity, helping to create a series of beneficial mechanical effects for treatment (such as increasing or decreasing volume air bubbles, and air bubbles push each other far away, increases the pharmaceuticals permeability in the walls of air bubbles/increases the contact between the pharmaceutical and pathogens and toxins in tissue disease . . . ), helps the treatment effectiveness for diseased tissues increase outstandingly, with much shorter treatment time, and helps reduce the recurrence;
causing pathogenic agents/pathogenic entities to locate in thin walls that formed by tiny air bubbles (for chelator-foam-strong physics effect pharmaceutical type), thereby increasing the dispersion speed of the active ingredients of the pharmaceutical in the diseased tissue with very high levels, and causing the active ingredients of the pharmaceutical to completely contact with all pathogenic agents/pathogenic entities in the treated tissues;
causes the active ingredients of the pharmaceutical to penetrate strongly into the pathogenic agents/pathogenic entities in the thin walls due to the continuous changes in pressure of the air bubbles (that create the thin walls), thanks to that, the amount of the active ingredients of the pharmaceutical to enter into pathogenic agents/pathogenic entities in the thin walls highly increases, quickly inactivating the pathogenic agents and quickly killing the pathogenic entities in these thin walls.

### Implementation Examples

Chelator pharmaceuticals to treat Covid 19 disease, treating patients with severe symptoms and patients who have not received any Covid 19 vaccine shots, patients are given the oral pharmaceutical (with each dose (adult dose) containing [1.4 g EDTA Mg (6% Mg type)]+2.6 g water+glycerol to make 5 ml)] (about a tea spoon), the drug is taken 3 doses per day, and take for 7 days, and from the 8th to the 15th day take ½ of the above dose every day (take for an additional from 8 days to prevent the appearance of post-Covid syndrome), the result of 4 hours after taking the first dose is the symptoms that are clearly reduced and the patients become much more pleasant, after 48 hours, the symptoms remains negligible, then after a few days later, the disease was cured, and after treatment, the patient did not have post-Covid 19 syndrome.
Chelator pharmaceutical to treat Covid 19 disease, treating the patients who are over 75-year-old with severe symptoms and with the lung infection and the patients who received her last Covid vaccine injection over 16 months ago, the patient received chelator pharmaceuticals as mentioned above, and took it in the same way as mentioned above, along with Bactrim at the dose is ¼ of the normal dose of Bactrim, and taking Bactrim for 7 days, the result is the Covid 19 disease and lung infection was cured after 7 days.

The chelator pharmaceutical to treat the influenza for the 63-year-old patient with severe symptoms, the patient was given the chelator pharmaceutical with each dose (adult dose) containing [1.4 g EDTA Mg (adult dose). 6% Mg)]+2.6 g water+glycerol enough 5 ml)] (about a tea spoon), the pharmaceutical is taken 3 doses per day, and taken for 7 days, 3 hours after taking the first dose, most of the symptoms were significantly reduced and the patient felt much better, and after 36 hours most symptoms were gone, and after 5 days the disease was over.

The chelator pharmaceutical o treat the viral fever for the 8-year-old patient with severe symptoms, the patient was given the chelator pharmaceutical with each dose (dose for children is about 30 kg) containing (½ the dose compared to adult dose) [0.7g EDTA Mg (6% Mg type)]+1.3 g water+glycerol enough 2.5 ml)] (about ½ a tea spoon), the pharmaceutical is taken 3 doses per day, and took for 7 days, the result is the patient's fever decreased and after 3 hours after taking the first dose, the patient returned to active play and eating as before disease suffering, and the disease is cured after 5 days.

The chelator pharmaceutical to treat the avian influenza in chickens suffering avian influenza (influenza virus strain is not undetermined), with the chicken flock has many individuals that were lethargic state and many chickens began to die, 10% of chicken flock were kept for control experiments and given normal water, 90% of chicken flock were given water with 3% Mg EDTA, results after 12 hours the chickens that drank water containing 3% Mg EDTA, they stopped the lethargic state and they returned to eating normally, while the control chickens continued the lethargic state and stopped eating.

The chelator pharmaceutical agent to treat the long Covid syndrome after recovering from Covid 19, with the case of long Covid syndrome is the 57-year-old man who had previously had Covid 19 and had received 3 shots of Covid 19 vaccine previously, with symptoms of walked up the stairs to the 4th floor, breathing much faster than before he suffered the Covid 19, after using for 4 weeks the chelator pharmaceutical with each dose (adult dose) containing [[0.3 g triethylenetetramine neutral glutamate+0.6 g EDTA Mg], and took it twice a day, after 4 weeks of taking the medicine, he walked up the stairs to the 4th floor without the rapid breathing, and breathing returns to normal like before Covid 19.

The chelator pharmaceutical to treat the toothache symptoms in toot cavities by using the cotton gauze pad wrapping around the powder of the chelator pharmaceutical containing [20% Ca EDTA powder+80% zeolite powder] and inserted into the tooth cavity, the result is that after about 10 minutes, the toothache symptoms gradually decrease and the pain disappears after about 15 minutes.

The chelator pharmaceutical to treat the symptoms of loss of taste in Covid 19 disease, by sucking the drug solution containing 2% Ca EDTA for a few minutes and then spit it, the results is the sweet taste to recover immediately afterwards, obvious recovery for the loss of taste of sweeteners generated by cane sugar/aspartame/sucralose.

The chelator pharmaceutical to treat the symptoms of sore throat/cough in diseases caused by microorganisms, by lozenging candy with each 2 g candy containing [0.3 g Ca EDTA+0.6 glycerol+1 g gelatine+water just enough 2 g], the result is that after sucking for about 10 minutes, the symptoms of sore throat/cough are reduced, after many times of sucking (a large number of these chelator pharmaceutical candies can be sucked for 24 hours, due to CA EDTA is highly safe when used in high doses (adults can use under 120 g of Ca EDTA in 24 hours with 20 g for each time and 4 hours apart, still have no side effects).

The chelator pharmaceutical to treat the symptoms of dry eyes/eye pain/red eyes, by instilling into the eyes the chelator pharmaceutical solution containing [0.1% triethylenetetramine diethylenetriaminepenatacetate (neutral)+enough 100% water) can be instilled from 2 to 6 times a day, the result is after a few minutes of instilling the above solution, the symptoms of dry eyes/eye pain will no longer exist and the symptoms of red eyes will decrease after a few hours.

The chelator pharmaceutical to treat the symptoms of headaches of unknown cause, by taking chelator pharmaceutical with each dose (adult dose) containing 0.5 g of triethylenetetramine diethylenetriaminepenatacetate (may be in pill form), the result is after about 2 hours, the headache symptoms will be significantly reduced.

The chelator pharmaceutical to treat the post-Covid syndrome with shortness of breath when exercising vigorously (such as going up stairs (which was not there before Covid) for the case of the 57-year-old male patient with Covid symptoms severe symptoms and without the use of the chelator pharmaceutical to treatthe Covid 19), the post-Covid syndrome of this patient was treated with the chelator pharmaceutical with each dose containing [0.5 g Mg EDTA+1 g glycerol+2 g of water], taking twice per day for 1 month, the result is that after 1 week, the shortness of breath during vigorous exercise is significantly reduced, and after 1 month of treatment, the shortness of breath during vigorous exercise is no longer present, and the treated person feels as pleasant as before Covid 19 disease.
The chelator pharmaceutical to treat the disease caused by toxin with the toxin is rat poison having the active ingredient being flocoumafen, feed the mice in cages the pills containing flocoumafen, and 12 hours later place in the mouse cage the cup of water solution having the chelator pharmaceutical containing the active ingredient being 0.3% triethylenetetramine diethylenetriaminepenatacetate, the result is that after many days the mice still lived, while in the control cage keeping the mice which were fed the above rat poison, but the water cup is just normal water, the result is all the mice died after about 2 days.

The chelator pharmaceutical to treat the the diseases caused by toxins/neurotoxins, by keeping Boraras Micros fish in water containing the chelator pharmaceutical with the active ingredient being triethylenetetramine diethylenetriaminepenatacetate, the clinical trials on animals (such as Borara Micros fish) were performed and achieved as the following results:
keeping fish in water with [5 ppm Nereistoxin+50-300 ppm triethylenetetramine diethylenetriaminepentaacetate], fish lived for many days (up to weeks), while keeping fish in water with 5 ppm Nereistoxin, fish died after 30 minutes;
keeping fish in water with [1.25 ppm flocoumafen (1/4 tablet of rat poison (with 0.005% flocoumafen) in 1 liter of water)+50-300 ppm triethylenetetramine diethylenetriaminepentaacetate], fish lived for days (up to weeks), while keeping fish In water with 1.25 ppm flocoumafen, fish died after 36 hours;
keeping fish in water with [0.3 ppm Bromadiolone+50-300 ppm triethylenetetramine diethylenetriaminepentaacetate], fish lived for many days, while keeping fish in water with 0.3 ppm Bromadiolone, fish died in 12 hours;
keeping fish in water with [0.2 ppm Methylamine Avermectin 50-300 ppm triethylenetetramine diethylenetriaminepentaacetate], fish lived for many days, while keeping fish in water with 0.2 ppm Methylamine Avermectin, fish died in 12 hours;
keeping fish in water with [2.5 ppm Emamectin benzoate 50-300 ppm triethylenetetramine diethylenetriaminepentaacetate], fish lived for many days, while keeping fish in water with 2.5 ppm Emamectin benzoate, fish died in a few hours;
keeping fish in water with [1.0 ppm phenylperazole+50-300 ppm triethylenetetramine diethylenetriaminepentaacetate], fish lived for days, while keeping fish in water with 1.0 ppm phenylperazole, fish died in a few hours;
keeping the fish in water with [[0.2 ppm Alpha Cypermethrin+50-300 ppm triethylenetetramine diethylenetriaminepentaacetate],], the fish lived for many days, while keeping the fish in water with 0.2 ppm Alpha Cypermethrin, fish died in a few hours.

The chelator-physics effect pharmaceuticals (the chelator-physics effect drugs) to treat the severe bacterial infection tissues (on human) that have been previously treated with multiple antibiotics (topical and oral) for over 1 week without improvement, these bacterial infection tissues are treated by applying the above pharmaceutical solution to the open wound on the bacterial infection tissues with 4-6 times/day for 7 days, and applying by the chelator-physics effect pharmaceuticals containing [0.4 % Cu EDTA+1.2 % Mg EDTA+2.4 % Ca EDTA+70 %+100 % water], the result is that after 24 hours the infection in treated tissues are significantly decreased/the inflammation in treated tissues are significantly decreased, after 3 days the treated tissues are completely without infection/inflammation, and after 7 days the open wound on the treated tissues are healed and the treated tissues are cured.

Pharmaceuticals to treat the dark-colored benign tumors under the skin (on humans) with a diameter of about 8 mm by applying to the open wound on the tumors (open wounds are created by making needle incisions on the surface of the tumors), and apply about 6 times/day for 10 days, and apply with chelator-strong physics effect pharmaceutical containing [0.4 % Cu EDTA+1.2 % Mg EDTA+2.4% Ca EDTA+0.5% triethylenetetramine diethylenetetraminepentaacetate+70% glycerol+just enough 100% water], the result is that after 3 days the tumor size is clearly reduced and a solid core in the middle of the treated tumor to appear, after 7 days this core becomes more solid and shrinks, after 19 days this solid core falls out of the tumor treatment. and after 10 days, the open wound heals and the surface of the tumor treated is flat as the flat of the skin surface, and leaves negligible scars.

The chelator-physical effect pharmaceutical to treat the melanoma tumor (on humans) with the diameter of about 12 mm, by injecting into the tumor the solution of the chelator-physical effect pharmaceutical] containing [0.4 % Cu EDTA+1.2 % Mg EDTA+2.4 % Ca EDTA+0.5 % triethylenetetramine diethylenetetraminepentaacetate+70 % glycerol+water just enough 100%], the result is similar to the results of treating dark-colored benign tumors as mentioned in above section.

The chelator-foam-physics effect pharmaceutical to treat the fungal-infection tissue (on human) are warts (the tissue proliferating by HPV viruses) with the size of 4 mm to 10 mm, by applying to the surface of these warts the chelator-foam-physics effect pharmaceutical containing [0.4 % Cu EDTA+1, 2 % Mg EDTA+2.5% triethylenetetramine diethylenetetraminepentaacetate+70% glycerol+10% H 202+enough 100% water], apply for 5 days, and apply 4-6 times per day, the result are that after 3 days the hardness dry core to appear in the middle of the treated tissues, after about 6 days this hardness dry core falls out of the treated tissues, and a few days later the surface of the treated tissues become flat as the flat of the skin surface around the treated tissue, and leaves almost no scars.

The chelator solution to treat the environmental with the composition containing [1% Ca EDTA+1% propylene glycol+100% water], the chelator solution is sprayed into the rooms having persons with Covid 19, can spray into the room space about 5 minutes before the caregivers entered the room and came into contact with the patient with Covid 19 (both were wearing masks, and the drug solution was also sprayed on the caregiver's mask, after 15 days the caregivers were not suffered the Covid 19 disease.

The chelator solution to treat the environmental with the composition containing [10% Ca EDTA+water just enough 100%], the solution is sprayed onto the water surface where fish clusters is floating and already to die in water channels with stagnant water/slow-flowing water, with the fishes are poisoned by toxins (water-soluble toxins) that accumulate on the land during the dry season, the results are that the above water surface areas that are treated as mentioned above, the fishes dived down after about 10 minutes, while in untreated water surface areas, the fish still float and a few hours later several of these floating fishes died.

The chelator cosmetic cream to treat the acne and improve skin properties with the composition containing [0.5% triethylenetetramine diethylenetetraminepentaacetate+10% glycerol+1% triethylenetetramine ricinoletate+2% triethylenetetramine stearate+enough 100% water] , the cream is used to apply to skin areas with acne/melasma (due to the accumulation of heavy metals, such as due to the accumulation of Hg by cosmetics containing Hg, or due to the accumulation of heavy metals naturally due to low concentration of polyamine derivatives in the skin for a long time)/dry skin areas/inelastic skin areas, the results are that after 1 week of applying the above cosmetic cream to the above mentioned skin areas and applying at least once a day, the acne will be significantly reduced/disappeared, and mechanical property of the skin/the moisture of the skin will be significantly improved, after 6 months, the melasma will be significantly reduced/disappeared.

The chelator solution to treat the environmental with the composition containing [3% Ca EDTA +water just enough 100%], the solution to treating the water contaminated with heavy metals in canals having fish/in ponds having fish where in the beginning of the rainy season, the water rich in heavy metal derivatives that are poured from the land into these canals/pond, causing fish to float and die, the result are that after 20 minutes after spreading the above the chelator solution evenly on the water surface where the fishes are floating, the fishes dive and the fishes does not die.

Food preservation chelator additive has the composition containing [2% triethylenetetramine diethylenetetraminepentaacetate+water just enough 100%], solution is sprayed on the surface of food/to mix into processed foods (such as pate) to prevent food that are kept for a long time to avoid the appear toxins/neurotoxins due to contamination with microorganisms that produce toxins/neurotoxins, due to triethylenetetramine diethylenetetraminepentaacetate inactivates toxins/neurotoxins in low concentration (over 30 ppm) while preventing the proliferation of organic mattes-decomposition microorganisms, therefore, when the processed foods in grining form are added with the above additives at concentrations above 30 ppm in processed foods, these additives will prevent the risk from poisoning for the user.

The chelator solution to wash the hands/to sanitize the surfaces/to spray into the environment where people live, the chelator solution has the composition containing [2% Ca EDTA+1% propylene glycol+70% alcohol+water just enough 100%], after the alcohol and water evaporate from the sterilized surface, the sterilized surfaces are left with [Ca EDTA+propylene glycol] which is strongly permeable to pathogenic microorganisms, and make the pathogenic microorganisms to immediately die right after the alcohol and water to evaporate from the sterilized surfaces with the above chelator solution.

The chelator additive to preserve blood (blood used for blood transfusions) which helps prevent blood from clotting and helps the transfused blood has the ability to resist bacteria or has the ability to treat diseases caused by microorganisms or has the ability to strengthen the immune system, the chelator additive has the composition containing triethylenetetramine diethylenetetraminepentaacetate, and this additive is added to the blood for transfusion, with the concentration of triethylenetetramine diethylenetetraminepentaacetate in the blood for transfusion is from 100 ppm to 1000 ppm.

The chelator solution to preserve the tissue awaiting the transplantation that has the composition containing [2000 ppm triethylenetetramine diethylenetetraminepentaacetate+30% glycerol+enough 100% water], this chelator solution is used for preservation of living tissues in environments with temperatures from 1 degree Celsius to 4 degrees Celsius waiting for a tissue transplant, the tissue prepared for transplantation is placed cold water at 1 degree Celsius until the glycerol concentration in the tissue drops below 10% before it can be transplanted, living tissue preservation solution made of triethylenetetramine diethylenetetraminepentaacetate and glycerol, therefore the biological structure of tissues are less damaged and the physical structure of the tissues are less damaged, so when transplanting theses tissues are less rejected and less infected, after transplantation, it is necessary for the patient to take chelator pharmaceuticals for a long enough time to help to avoid the graft rejection.

The chelator solution to use in open surgery that has the composition containing [500 ppm triethylenetetramine triethylenetetraminepentaacetate+20% glycerol+5% propylene glycol+sufficient water], the solution is applied to exposed tissues where open surgery is performed, this chelator solution will help prevent the infection for open wounds, and helps wounds after stitching after surgery to heal quickly (experiments of applying the above solution to open wounds under the skin with infection, makes the infection go away quickly and especially reduces pain and makes the treated tissue quickly recover, thanks to the glycerol makes the treated tissue to be wet, and chelator cleans the treated tissue from the accumulation of Zn ions/other transition metal ions which help the treated tissue to recover quickly).

1. Pharmaceuticals containing chelators/combinations of chelators, with chelators that have the effect of holding Zn ion/other transition metal ions, in which chelators are salts formed from polyamine with aminopolycarboxylic acid/salts formed from aminopolyol with aminopolycarboxylic acid/salts formed from polyamine with acid having at least 2 carboxyl functional groups in the molecule/salts formed from polyamine with acid having at least 2 hydroxyl groups in molecule/salts formed from polyamine and acid having at least 1 sulfur-containing functional group in the molecule/salts formed from polyamine with ascorbic acid/Mg aminopolycarboxylate salt/Ca aminopolycarboxylate salt/Cu aminopolycarboxylate salt/Fe aminopolycarboxylate salt/Ag aminopolycarboxylate salt/other aminopolycarboxylic acid derivatives in which other aminopolycarboxylic acids participate to form the derivatives of aminopolycarboxylic acids are aminopolycarboxylic acids with at least 3 carboxyl groups in the molecule/other derivatives of aminopolycarboxylic acids in which aminopolycarboxylic acids participate in forming other derivatives of aminopolycarboxylic acid that are aminopolycarboxylic acids having at least 3 carboxyl-containing groups in the molecule/other derivatives of polyamines in which polyamines participate in forming other polyamine derivatives that are polyamines having at least 4 nitrogen-containing functional groups in the molecule, and the pharmaceuticals are used for the following purposes:
to prevent or to treat symptoms such as itchy throat symptoms/sore throat symptoms/inflamed throat symptoms/cough symptoms/fever symptoms/loss of taste symptoms/fatigue symptoms/myalgia symptoms/joint pain symptoms/anorexia symptoms/rash symptoms/cytokine storm symptoms/headache symptoms/chills symptoms/decreased cognitive symptoms/dyspnea symptoms/respiratory distress symptoms/acute respiratory failure symptoms/other symptoms caused by excessive accumulation of Zn ions or other heavy metal ions in tissues/nerve tissues/brain tissue;
or to prevent or to treat diseases caused by pathogenic microorganisms that are accompanied by symptoms such as itchy throat symptoms/sore throat symptoms/inflamed throat symptoms/cough symptoms/fever symptoms/loss of taste symptoms/fatigue symptoms/myalgia symptoms/joint pain symptoms/anorexia symptoms/rash symptoms/cytokine storm symptoms/headache symptoms/chills symptoms/decreased cognitive symptoms/dyspnea symptoms/respiratory distress symptoms/acute respiratory failure symptoms/other symptoms caused by excessive accumulation of Zn ions or other heavy metal ions in tissues/nerve tissues/brain tissue;
or to prevent or treat diseases caused by toxins or neurotoxins, in which toxins or neurotoxins are toxins or neurotoxins produced by pathogenic microorganisms in the host body, or in which toxins or neurotoxins are external toxins or external neurotoxins, with external toxins or external neurotoxins that can be pesticides/rat poisons or other animal killers/venoms of venomous animals/poisonous mushrooms/poisonous plants/organisms whose body parts contain toxins/toxins produced by biotechnology/organic toxins or inorganic toxins;
or to prevent or to treat post-disease syndromes due to the accumulation of Zn ion/Zn ion-other heavy metal ion complexes in tissues/nerve tissues/brain tissues of the host during the host with disease caused by pathogenic microorganisms, or due to the accumulation of Zn ion/Zn ion-other heavy metal ion complexes in tissues/nerve tissues/brain tissues of the host causing by the low concentration of polyamine derivatives in the blood of the host for long periods of time;
or to prevent or to treat post-disease syndromes which appear after the host suffers from diseases caused by pathogenic microorganisms;
or to prevent or to treat syndromes in the elderly, or to strengthen the immune system in the elderly;
or to strengthen weakened immune systems in chronic diseases;
or to combine with other active treatment ingredients to form the pharmaceutical type with higher treatment effects or to form the pharmaceutical type with the low content of another treatment active ingredient;
or to combine with antibiotic active ingredients of to form pharmaceutical type with higher treatment effectiveness than the treatment effectiveness of antibiotic active ingredients, or to form pharmaceutical type that has the low contents of antibiotic active ingredients;
or to combine with polyols with the concentrations of polyols is greater than 20% to form a pharmaceutical type to treat diseased tissues, with diseased tissues that can be bacterial infection tissue/necrotic tissues/fungal-infection tissues/tissue contains fluid with many toxins/dark color-benign tumors/cancerous tissue,

2. Pharmaceuticals with ingredients and effects and are used for the purposes according to claim 1, in which polyamine derivatives are made from polyamines with at least 4 nitrogen-containing functional groups in the polyamine molecule having at least 4 nitrogen-containing functional group in the molecule is Triethylenetetramine/Tetraethylenepentamine/Pentaethylenehexamine/ Higher ethylenepolyamines/N-(2-hydroxyethyl)ethylenediamine/Polyethylene polyamines/Polyethyleneimine/Branched Polyethyleneimine/1,1,1-Tris(aminomethyl)ethane/ poly(ethylenimine) ethoxylated/polylysine/polyarginine/poly(amidoamine).

3. Pharmaceuticals with ingredients and effects and are used for the purposes according to claim 1, in which aminopolycarboxylic acid has at least 3 carboxyl-containing functional groups in the molecule to participate in forming aminopolycarboxylic acid derivatives that aminopolycarboxylic acid has at least 3 carboxyl groups in the molecule is Ethylenediaminetetraacetic acid/Diethylenetriaminepentaacetic acid/Triethylenetetramine-N, N, N′, N″, N′″, N′″-hexaacetic acid/1,2-cyclohexylenediamine tetra-acetic acid/Dibenzothiophene-1,3,6,8-Tetracarboxylic acid/N-hydroxyethyl-ethylenediamine-triacetic acid/trimethylenediaminetetraacetic acid/ethylene glycol bis(2-aminoethyl ether)-N, N, N′, N′-tetraacetic acid/1,2-bis(2-aminophenoxy)ethane-N, N, N′, N′-tetra-acetic acid/1,4,7-triazacyclononane-1,4,7-triacetic acid/1,4,7,10-tetraazacyclododecane-1,4,7,10-tetraacetic acid/N, N′-bis(2-hydroxyethyl)ethylenediamine-N, N′-diacetic acid/1,4,7,10,13-pentaazocyclopentadecane pentaacetic acid/1,4,7,10,13,16-hexaazocyclooctadecane hexaacetic acid/1,4,8,11-tetraazacyclotetradecane-1,4,8,11-tetraace-tic acid/triethylenetetraminepentaacetic acid/4,5-diphenyl-1,3-oxazol-2-yl)thio](phenyl)acetic acid/1,4,7,10-tetraaxacyclododecane-1,4,7,10-tetramethylenephosphonic acid/Ethylenediamine-N, N′-disuccinic acid/2-hydroxyethyliminodiacetic acid/Glutamic acid diacetate/Methylglycinediacetic acid/1-aspartic acid N, N-diacetic acid/ethylenediamine-N, N′-bis(2-hydroxyphenylacetic acid/ethylene glycol bis(2-aminoethyl ether)-N, N, N′, N′-tetraacetic acid /[2-{4,7-biscarboxymethyl(1,4,7)triazacyclonona-1-yl-ethyl} carbonylmethylamino] acetic acid /1,2-cyclohexylenedinitrilotetraacetic acid/O, O'-bis(2-aminoethyl)ethyleneglycol-N, N, N′, N′-tetraacetic acid]/1,4,7,10-Tetraazacyclododecane-1,4,7,10-tetraacetic acid/6-amino-6-methylperhydro-1,4-diazepinetetraacetic acid.

4. Pharmaceuticals with ingredients and effects and are used for the purposes according to claim 1, in which salt is formed from polyamine with aminopolycarboxylic acid being formed polyamine which polyamine is lysine/arginine/spermidine/spermine/Triethylenetetramine/ Tetraethylenepentamine/Pentaethylenehexamine/Higher ethylenepolyamines/N-(2-hydroxyethyl)ethylenediamine/Polyethylene polyamines/Polyethyleneimine/Branched Polyethyleneimine/1,1,1-Tris(aminomethyl)ethane/ethoxylated poly (ethylenimine)/polylysine/polyarginine/poly(amidoamine ) with aminopolycarboxylic acid which aminopolycarboxylic acid is Ethylenediaminetetraacetic acid/Diethylenetriaminepentaacetic acid/Triethylenetetramine-N, N, N′, N″, N′″, N′″-hexaacetic acid/1,2-cyclohexylenediamine tetra-acetic acid/Dibenzothiophene-1,3,6,8-Tetracarboxylic acid/N-hydroxyethyl-ethylenediamine-triacetic acid/trimethylenediaminetetraacetic acid/ethylene glycol bis(2-aminoethyl ether)-N, N, N′, N′-tetraacetic acid/1,2-bis(2-aminophenoxy)ethane-N, N, N′, N′-tetra-acetic acid/1,4,7-triazacyclononane-1,4,7-triacetic acid/1,4,7,10-tetraazacyclododecane-1,4,7,10-tetraacetic acid/N, N′-bis(2-hydroxyethyl)ethylenediamine-N, N′-diacetic acid/1,4,7,10,13-pentaazocyclopentadecane pentaacetic acid/1,4,7,10,13,16-hexaazocyclooctadecane hexaacetic acid/1,4,8,11-tetraazacyclotetradecane-1,4,8,11-tetraace-tic acid/triethylenetetraminepentaacetic acid/4,5-diphenyl-1,3-oxazol-2-yl)thio](phenyl)acetic acid/1,4,7,10-tetraaxacyclododecane-1,4,7,10-tetramethylenephosphonic acid/Ethylenediamine-N, N′-disuccinic acid/2-hydroxyethyliminodiacetic acid/Glutamic acid diacetate Methylglycinediacetic acid/1-aspartic acid N, N-diacetic acid/ethylenediamine-N, N′-bis(2-hydroxyphenylacetic acid/ethylene glycol bis(2-aminoethyl ether)-N, N, N′, N′-tetraacetic acid/[2-{4,7-biscarboxymethyl(1,4,7)triazacyclonona-1-yl-ethyl} carbonylmethylamino] acetic acid/1,2-cyclohexylenedinitrilotetraacetic acid/O, O′-bis(2-aminoethyl)ethyleneglycol-N, N, N′, N′-tetraacetic acid]/1,4,7,10-Tetraazacyclododecane-1,4,7,10-tetraacetic acid/6-amino-6-methylperhydro-1,4-diazepinetetraacetic acid.

5. Pharmaceuticals with ingredients and effects and are used for the purposes according to claim 1, in which derivatives of aminopolycarboxylic acid is Mg aminopolycarboxylate/Ca aminopolycarboxylate/Cu aminopolycarboxylate/Fe aminopolycarboxylate/Ag aminopolycarboxylate which is formed from Mg derivative/Ca derivative/Cu derivative/Fe derivative/Ag derivative with aminopolycarboxylic acid which aminopolycarboxylic acid is Ethylenediaminetetraacetic acid/Diethylenetriaminepentaacetic acid/Triethylenetetramine-N, N, N′, N″, N′″, N′″-hexaacetic acid/1,2-cyclohexylenediamine tetra-acetic acid/Dibenzothiophene-1,3,6,8-Tetracarboxylic acid/N-hydroxyethyl-ethylenediamine-triacetic acid /trimethylenediaminetetraacetic acid/ethylene glycol bis(2-aminoethyl ether)-N, N, N′, N′-tetraacetic acid/1,2-bis(2-aminophenoxy)ethane-N, N, N′, N′-tetra-acetic acid/1,4,7-triazacyclononane-1,4,7-triacetic acid/1,4,7,10-tetraazacyclododecane-1,4,7,10-tetraacetic acid/ N, N′-bis(2-hydroxyethyl)ethylenediamine-N, N′-diacetic acid/1,4,7,10,13-pentaazocyclopentadecane pentaacetic acid/1,4,7,10,13,16-hexaazocyclooctadecane hexaacetic acid/1,4,8,11-tetraazacyclotetradecane-1,4,8,11-tetraace-tic acid/triethylenetetraminepentaacetic acid/4,5-diphenyl-1,3-oxazol-2-yl)thio](phenyl)acetic acid/1,4,7,10-tetraaxacyclododecane-1,4,7,10-tetramethylenephosphonic acid/Ethylenediamine-N, N′-disuccinic acid/2-hydroxyethyliminodiacetic acid/Glutamic acid diacetate/Methylglycinediacetic acid/1-aspartic acid N, N-diacetic acid/ethylenediamine-N, N′-bis(2-hydroxyphenylacetic acid/ethylene glycol bis(2-aminoethyl ether)-N, N, N′, N′-tetraacetic acid/[2-{4,7-biscarboxymethyl(1,4,7)triazacyclonona-1-yl-ethyl} carbonylmethylamino] acetic acid/1,2-cyclohexylenedinitrilotetraacetic acid/O, O′-bis(2-aminoethyl)ethyleneglycol-N, N, N′, N′-tetraacetic acid]/1,4,7,10-Tetraazacyclododecane-1,4,7,10-tetraacetic acid/6-amino-6-methylperhydro-1,4-diazepinetetraacetic acid.

6. Pharmaceuticals with ingredients and effects and are used for the purposes according to claim 1, in which salts is formed from polyamines with acids having at least 2 carboxyl-containing functional groups in the molecule being formed from polyamine is lysine/arginine /spermidine/spermine/Triethylenetetramine/Tetraethylenepentamine/Pentaethylenehexamine/ Higher ethylenepolyamines/N-(2-hydroxyethyl)ethylenediamine/Polyethylene polyamines/Polyethyleneimine/Branched Polyethyleneimine/1,1,1-Tris(aminomethyl)ethane/ ethoxylated poly(ethylenimine)/polylysine/polyarginine/poly(amidoamine) with acids that have at least 2 carboxyl functional groups in the molecule and acids that have at least 2 carboxyl functional groups in the molecule are fumaric acid/itaconic acid/glutamic acid/aspartic acid/carbonic acid/phosphoric acid/citric acid/succinic acid/malic acid.

7. Pharmaceuticals with ingredients and effects and are used for the purposes according to claim 1, in which salts is formed from polyamine and acids that have at least one sulfur-containing functional group in the molecule being formed from polyamine is lysine/arginine/spermidine/spermine/Triethylenetetramine/ Tetraethylenepentamine/Pentaethylenehexamine/Higher ethylenepolyamines/N-(2-hydroxyethyl)ethylenediamine/Polyethylene polyamines/Polyethyleneimine/Branched Polyethyleneimine/1,1,1-Tris(aminomethyl)ethane/poly(ethylenimine) are ethoxylated /polylysine/polyarginine/poly(amidoamine) with acid having at least 1 sulfur-containing functional group in the molecule and acid having at least 1 sulfur-containing functional group in the molecule is sulfonic acid/sulfuric acid/sulfurous acid/sulfenic acid/sulfinic acid/hydrogen sulfide acid/hyposulfurous acid.

8. Pharmaceuticals with ingredients and effects and are used for the purposes according to claim 1, in which salt is formed from polyamines with acid having at least 2 hydroxyl-containing functional groups in the molecule being polyamines which polyamines is lysine/arginine /spermidine/spermine/Triethylenetetramine/Tetraethylenepentamine/Pentaethylenehexamine/ Higher ethylenepolyamines/N-(2-hydroxyethyl)ethylenediamine/Polyethylene polyamines/Polyethyleneimine/Branched Polyethyleneimine/1,1,1-Tris(aminomethyl)ethane/ ethoxylated poly(ethylenimine)/polylysine/polyarginine/poly(amidoamine) with acids having at least 2 hydroxyl-containing functional groups in the molecule, acids with at least 2 hydroxyl-containing functional groups in the molecule is ascorbic acid/gluconic acid/gallic acid/tannic acid.

9. Pharmaceuticals with ingredients and effects and are used for the purposes according to claim 1, in which salt is formed from aminopolyol with aminopolycarboxylic acid being salt formed from aminopolyol of which aminopolyol is
Monoethanolamine/Diethanolamine/Triethanolamine/2-amino-2-methyl-1-propanol/Methyldiethanolamine
with aminopolycarboxylic acid mà aminopolycarboxylic acid is
Ethylenediaminetetraacetic acid/Diethylenetriaminepentaacetic acid/Triethylenetetramine-N, N, N′, N″, N′″, N′″-hexaacetic acid/1,2-cyclohexylenediamine tetra-acetic acid/Dibenzothiophene-1,3,6,8-Tetracarboxylic acid/N-hydroxyethyl-ethylenediamine-triacetic acid/trimethylenediaminetetraacetic acid/ethylene glycol bis(2-aminoethyl ether)-N, N, N′, N′-tetraacetic acid/1,2-bis(2-aminophenoxy)ethane-N, N, N′, N′-tetra-acetic acid/1,4,7-triazacyclononane-1,4,7-triacetic acid/1,4,7,10-tetraazacyclododecane-1,4,7,10-tetraacetic acid/ N, N′-bis(2-hydroxyethyl)ethylenediamine-N, N′-diacetic acid/1,4,7,10,13-pentaazocyclopentadecane pentaacetic acid/1,4,7,10,13,16-hexaazocyclooctadecane hexaacetic acid/1,4,8,11-tetraazacyclotetradecane-1,4,8,11-tetraace-tic acid/triethylenetetraminepentaacetic acid/4,5-diphenyl-1,3-oxazol-2-yl)thio](phenyl)acetic acid/1,4,7,10-tetraaxacyclododecane-1,4,7,10-tetramethylenephosphonic acid/Ethylenediamine-N, N′-disuccinic acid/2-hydroxyethyliminodiacetic acid/Glutamic acid diacetate/Methylglycinediacetic acid/1-aspartic acid N, N-diacetic acid/ethylenediamine-N, N′-bis(2-hydroxyphenylacetic acid/ethylene glycol bis(2-aminoethyl ether)-N, N, N′, N′-tetraacetic acid /[2-{4,7-biscarboxymethyl(1,4,7)triazacyclonona-1-yl-ethyl} carbonylmethylamino] acetic acid /1,2-cyclohexylenedinitrilotetraacetic acid/O, O′-bis(2-aminoethyl)ethyleneglycol-N, N, N′, N′-tetraacetic acid]/1,4,7,10-Tetraazacyclododecane-1,4,7,10-tetraacetic acid/6-amino-6-methylperhydro-1,4-diazepinetetraacetic acid.

10. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9, in which the pharmaceutical ingredients include Mg derivatives/Ca derivatives/Cu derivatives/Fe derivatives/Zn derivatives/polyol/surfactants/Boron derivatives/peroxide.

11. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9 or according to claim 10, in which pharmaceuticals contain at least one additional treatment active ingredient that is different from treatment active ingredients in claim 1.

12. Pharmaceuticals with ingredients and effects and are used for the purposes according to claim 11, in which other treatment active ingredient is antibiotic active ingredient/antifungal active ingredient/anti-inflammatory active ingredient/analgesic active ingredient/active ingredient that reduces soft tissue pain/active ingredient that reduces bone tissue pain/active ingredient that reduces joint tissue pain/active ingredient that reduces headache/active ingredient that kills cancer cells.

13. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9 or according to claim 10 or according to claim 11, in which pharmaceuticals in solution form and containing polyols/and combinations of polyols with polyol that is glycerol/propylene glycol/sorbitol/mannitol/xylitol/arabitol/ribitol/erythriol/polyvinyl alcohol/polyethylene glycol/polybutylene glycol/polypropylene glycol/polyethylene glycol/polypropylene glycol, with the concentration of polyols in the pharmaceutical solution is greater than 10%, and the pharmaceutical solution is used to treat diseased tissues, with diseased tissues that may be bacterial infection tissues/necrotic tissues/fungal infection tissues/tissues containing fluid with many toxins/benign tumors/cancerous tumors.

14. Pharmaceuticals with ingredients and effects and are used for the purposes according to claim 13, in which the pharmaceutical solutions have glycerol concentration being greater than 30%.

15. Pharmaceuticals with ingredients and effects and are used for the purposes according to claim 10, in which the peroxide in the pharmaceuticals is H2O2/urea peroxide/MEK peroxide.

16. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9 or according to claim 12 or according to claim 13 or according to claim 14 or according to claim 15, in which pharmaceuticals are used for humans or for domestic animals or for wild animals.

17. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9 or according to claim 10, in which the pharmaceuticals are combined with water/with the solution containing NaCl/other liquid to form the liquid product for use by intramuscular/intravenous injection/arterial injection/intravenous infusion/arterial infusion/administration into the rectum/spray into the nose/spray into the alveoli/drop into the eyes/spray or apply to the skin/spray or apply to the wound/spray or apply to infected tissues with open wounds, or pharmaceuticals are created in the form of chelator pharmaceutical candy for suckibf/for insertion into the rectum.

18. Pharmaceuticals with ingredients and effects and are used for the purposes according to claim claim 1 or according to claim 2 or according to claim 3 or according to claim 4 or according to claim 5 or according to claim 6 or according to claim 7 or according to claim 8 or according to claim 9 or according to claim 10, in which pharmaceuticals are indicated for use during the use of other pharmaceuticals.

19. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9 or according to claim 10, in which the pharmaceuticals are prescribed for use at least two period per year, with each period at least 4 months apart.

20. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9, in which the pharmaceuticals are introduced into the beverages/food/processed food/drinking water/spice/vitamin supplements so that the active ingredients of pharmaceuticals are present in the host bodies regularly to help the host's immune system to strengthen/to reduce the accumulation of Zn ions and other heavy metal ions in the host bodies/to improve host health, with host are humans/animals.

21. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9, in which pharmaceuticals are introduced into cosmetics to reduce the accumulation of Zn ions/other transition metal ions on the skin and to prevent microorganisms to grow on the skin and to improve the biological/chemical/physical properties of skin.

22. Pharmaceuticals with ingredients and effects and are used for the purposes according to claim 21, in which cosmetics containing pharmaceuticals have added salts that are formed from polyamine with fatty acids/ricinooleic acid/arylsulfonic/alkylsulphonic acid.

23. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9 or according to claim 10, in which the pharmaceutical is combined with water to form an environmental treatment solution/introduced into the environmental treatment solution to form the solutions containing the active ingredients of the pharmaceuticals for environmental treatment.

24. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9, in which pharmaceuticals are introduced to body hygiene products, with body hygiene products can be mouthwash/eye drops/shampoo/shower oil/bath soap/hand soap/hand sanitizer/liquid alcohol disinfectant solution.

25. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9, in which pharmaceuticals are combined with water/other liquids to use to preserve living tissues waiting for transplantation.

26. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9, in which pharmaceuticals are used to preserve blood/artificial blood for blood transfusion, and at the same time so that blood/artificial blood has the ability to prevent and to treat diseases as claim 1.

27. Pharmaceuticals with ingredients and effects and are used for the purposes according to claim 1 or according to claim 2 or according to claim 3 or according to claim 4 or according to claim 5 or according to claim 6 or according to claim 7 or according to claim 8 or according to claim 9 or claim 10 or claim 11 or claim 12 or claim 13 or claim 14, in which pharmaceuticals are used in surgeries to prevent blood clotting/to prevent infection in surgical wounds.
